(19) United States
(12) Patent Application Publication
(10) Pub. No.: US 2026/0224762 A1
(43) Pub. Date: Aug. 6, 2026

(54) RADIONUCLIDE LABELLED PEPTIDE CONJUGATE FOR SITE-SPECIFIC UPAR-TARGETING

(71) Applicant: CURASIGHT A/S, Copenhagen N (DK)

(72) Inventor: Andreas KJÆR, Frederiksberg (DK)

(73) Assignee: CURASIGHT A/S, Copenhagen N (DK)

(21) Appl. No.: 19/148,530

(22) PCT Filed: Jan. 18, 2024
(86) PCT No.: PCT/EP2024/051181

§ 371 Date: Jul. 16, 2025

(30) Foreign Application Priority Data
Jan. 18, 2023 (EP) 23152283.0

(51) Int. Cl. A61K 51/08; A61P 35/00

(52) U.S. Cl. CPC A61K 51/088; A61P 35/00; A61K 2121/00

(57) ABSTRACT

The present invention provides a radionuclide labelled peptide for site-specific targeting of the Urokinase Plasminogen Activator Receptor (uPAR) and treatment of cancer diseases associated with high uPAR expression.

### FIELD OF THE INVENTION

The present invention relates to a radionuclide labelled peptide for site-specific targeting of the Urokinase Plasminogen Activator Receptor (uPAR) and treatment of cancer diseases associated with high uPAR expression.

### BACKGROUND OF THE INVENTION

Various radionuclide labelled peptide compositions have been developed or are under development for site-specific targeting of a therapeutic radionuclide. The general principle involves attaching a selected radionuclide to a peptide having a high specificity for a particular organ or tissue so that the organ or tissue can be treated by a therapeutic radioisotope. This field of research has shown promising applicability for tumor treatment.

Malignant tumors are capable of degrading the surrounding extracellular matrix, resulting in local invasion or metastasis. Urokinase-type plasminogen activator (uPA) and its cell surface receptor (uPAR) are central molecules for cell surface-associated plasminogen activation both in vitro and in vivo. uPAR is overexpressed in a variety of human cancers whereas the expression in non-cancer tissue is low and correlates with malignant tumor growth and is associate with a poor prognosis in many types of human cancers, possibly indicating a causal role for the uPA/uPAR system in cancer progression and metastasis. Studies by immunohistochemistry and in situ hybridization indicate that expression levels of the components from the uPA system are generally very low in normal tissues and benign lesions. It has also been reported that the uPA/uPAR system is involved in regulating cell-extracellular matrix interactions by acting as an adhesion receptor for vitronectin and by modulating integrin function. Based on these properties the uPA/uPAR system is consequently considered an attractive target for cancer therapy, and uPAR binding peptides including the peptide AE105 have been developed for targeting of the uPA/uPAR system such as described in U.S. Pat. No. 7,026,282. Furthermore, the radionuclide labelled uPAR targeting peptide conjugate 177Lu-DOTA-AE105 has been developed in the pursue of providing targeted radiotherapy for uPAR expressing cancers as described in WO 2013/167130. However, the tumor binding, as well as tumor retention of this peptide conjugate, was vastly decreased compared to that of the peptide alone, indicating that the AE105 peptide is not well suited for labeling with therapeutic radionuclide and has limited capabilities for providing targeted radiotherapy.

Development of therapeutic radionuclide labelled peptide conjugates is a complex process posing various challenges such as the stability of the conjugate which can be challenged by e.g. radiolysis by the therapeutic radionuclides and insufficient binding of the radionuclide within the chelator in a given conjugate structure. Furthermore, and particularly for therapeutic applications, target specificity is critical to achieve low toxicity as well as a therapeutic effect. Also retention of the conjugate at target is important for achieving optimal therapeutic effect. Despite extensive research pursuing peptide conjugates targeting various molecules, only two such conjugates have been approved for therapy, that is 177Lu-DOTA-TATE (Lutathera) and 177Lu-DOTA-PSMA (Pluvicto) for treatment of gastroenteropancreatic neuroendocrine tumors (GEP-NETs), which are positive for the hormone receptor somatostatin and metastatic castration-resistant prostate cancer (PSMA-positive mCRPC), respectively. Thus, there is a need for a stable radionuclide labelled uPAR targeting conjugate providing sufficient tumor binding and good tumor retention, so that cancer diseases associated with high uPAR expression can be treated with targeted radiotherapy.

### SUMMARY OF THE INVENTION

Surprisingly the inventor found that a 67Cu-DOTA-AE105 conjugate showed approximately 50 times higher tumor binding compared to 177Lu-DOTA-AE105 as well as much longer tumor retention (cf. example 5). Furthermore, 67Cu-DOTA-AE105 was found to be therapeutically effective in preclinical testing in a tumor mouse model (cf. example 4) whereas 177Lu-DOTA-AE105 did not show any therapeutical effect in the same tumor mouse model. 67Cu-DOTA-AE105 showed therapeutical effect in two further preclinical testing in tumor mouse models (cf. example 6 and example 7) confirming that uPAR targeting peptide based radionuclide conjugates and in particularly 67Cu-DOTA-AE105 are generally therapeutically effective across different types of uPAR expressing cancers. Furthermore, the inventor found that a 225Ac-DOTA-AE105 conjugate showed approximately 5 times higher tumor binding compared to 177Lu-DOTA-AE105, therapeutic efficacy in a glioblastoma xenograft tumor mouse model and showed promising results in tolerability test. Thus, even though earlier work had rejected the AE105 peptide as a suitable basis for future development of targeted radiotherapy of uPAR expressing cancers, the inventor successfully developed therapeutically effective radionuclide labelled uPAR binding conjugates based on the AE105 peptide in conjunction with both an alpha emitting radionuclide as well as a beta emitting radionuclide.

The radionuclide labelled peptide conjugates of the present invention having a high tumor binding and good tumor retention, offers a much wider therapeutic window in which the dose providing the optimal balance of therapeutic effect and side effects can be achieved compared to inferior counterparts.

In a first aspect, the present invention relates to a radionuclide labelled uPAR binding peptide conjugate, comprising
a) A uPAR binding peptide
b) A chelating agent suitable for binding radiometals
c) A radionuclide selected from the group consisting of 67Cu, 225Ac, 212Pb, 161Tb, and 149Tb
wherein the peptide is coupled to the radionuclide by the chelating agent.

Providing superior radionuclide labelled uPAR binding peptide conjugates suitable for treatment of uPAR expressing cancer diseases.

For the purpose of completeness, it is herewith clarified that the radionuclide labelled uPAR binding peptide conjugates according to the first aspect and all the embodiments thereof are preferably consisting of the specified components. That is, in a preferred form of the first aspect, the present invention relates to a radionuclide labelled uPAR binding peptide conjugate, consisting of
a) A uPAR binding peptide
b) A chelating agent suitable for binding radiometals
c) A radionuclide selected from the group consisting of 67Cu, 225Ac, 212Pb, 161Tb, and 149Tb
wherein the peptide is coupled to the radionuclide by the chelating agent.

In a second aspect, the present invention relates to a radionuclide labelled uPAR binding peptide conjugate according to the first aspect for use as a medicament.

In a third aspect, the present invention relates to a radionuclide labelled uPAR binding peptide conjugate according to the first aspect for use in the treatment of uPAR expressing cancer.

In a fourth aspect, the present invention relates to a method of treatment of a uPAR expressing cancer disease by administering to a patient a radionuclide labelled uPAR binding peptide conjugate according to the first aspect.

### BRIEF DESCRIPTION OF THE DRAWINGS

FIG. 1: shows 67Cu-DOTA-AE105 biodistribution in mice with xenograft tumors (U87MG).

FIG. 2: shows SPECT/CT images of 67Cu-DOTA-AE105 biodistribution in mice with human xenograft tumors (U87MG). More specifically, FIG. 2a shows axial, coronal, and maximum intensity projection (MIP) images of one representative animal from group A (30 MBq), and FIG. 2b: shows, axial, coronal and maximum intensity projection (MIP) images of one representative animal from group B (60 MBq).

FIG. 3: shows binding specificity of 67Cu-DOTA-AE105 at the uPAR expressing tumor.

FIG. 4: shows treatment efficacy of 67Cu-DOTA-AE105 in a glioblastoma mouse model.

FIG. 5: shows Kaplan-Meier curves of survival data from the treatment efficacy study of 67Cu-DOTA-AE105 in a glioblastoma mouse model.

FIG. 6: shows treatment efficacy of 177Lu-DOTA-AE105 in a human xenograft glioblastoma (U87MG) mouse model.

FIG. 7: shows treatment efficacy of 67Cu-DOTA-AE105 in a human xenograft glioblastoma mouse model (U87.MG) and human xenograft non-small cell lung cancer mouse model (NCI-1993).

FIG. 8: shows therapeutic effect of 67Cu-DOTA-AE105 in a human xenograft colorectal cancer mouse model.

FIG. 9: shows 67Cu-DOTA-AE105 induced apoptosis and DNA double strand breaks in treatment of a human xenograft colorectal cancer mouse model.

FIG. 10: shows 225Ac-DOTA-AE105 biodistribution and binding specificity in mice with human xenograft tumors (U87MG).

FIG. 11: shows tolerability of 225Ac-DOTA-AE105.

FIG. 12: FIG. 12a shows representation of HPLC chromatogram of the 30 kBq/nmol 225Ac-DOTA-AE105 based on gamma counted HPLC fractions. FIG. 12b shows Representation of HPLC chromatogram of the 40 kBq/nmol Ac-225-DOTA-AE105 based on gamma counted HPLC fractions.

FIG. 13: shows 225Ac-DOTA-AE105 induced apoptosis in glioblastoma xenograft tumor.

### DETAILED DESCRIPTION OF THE INVENTION

The radionuclide labelled uPAR binding peptide conjugates according to the first aspect of the present invention comprising a uPAR binding peptide, a chelating agent suitable for binding radiometals, and a radionuclide selected from the group consisting of 67Cu, 225Ac, 212Pb, 161Tb, and 149Tb, wherein the peptide is coupled to the radionuclide by the chelating agent, are stable conjugate structures having high specificity towards uPAR and good retention capabilities, thereby allowing for treatment of uPAR expressing cancers, in particular cancer types having high expression of uPAR.

uPAR Binding Peptides

The uPAR binding peptides of the present invention may be in the form of a monomeric peptide, i.e., a single peptide or a multimeric peptide construct such as a dimer, trimer or tetramer. A multimeric peptide construct may be homomeric (i.e., consisting of identical peptides) or heteromeric (i.e., consisting of non-identical uPAR binding peptides) and multimeric peptide construct can possess higher affinity for a target receptor than its monomeric equivalent.

The uPAR binding peptides of the present invention may be synthesized using standard methods. One example of a standard method is described in U.S. Pat. No. 7,026,282 as follows: the chain elongation steps of the Solid-phase peptide synthesis were carried out manually using polyethylene Syringes as reaction vessels. Synthesis was performed on KA-resin with preloaded Fmoc-amino acids on the acid labile linker. Five equivalents of Fmoc-amino acids activated by 1-hydroxy-7-benzotriazole (HOBt) and N,N′-diiso-propylcarbodiimide were used in the coupling steps and were allowed to react for more than 2 h. The Fmoc protecting group was removed with 20% piperidine in dimethyl formamide for 15-20 min. For cleavage, peptide-resins were treated for 1.5 h with 85% TFA containing 5% of phenol, mercaptoethanol, and thioanisole, respectively. The filtrates were concentrated by nitrogen flushing, and peptides were subsequently precipitated from, and washed four times with, diethyl ether. Peptides were finally dissolved/suspended in glacial acetic acid, lyophilised, redissolved in 10% acetic acid, and lyophilised again. Analytical HPLC analysis was performed on a Cs column using Waters 600E equipped with Waters photodiode array detector. A 25 min linear gradient from buffer A (0.1% TFA, 9.9% HO, 90% CHCN), was used. If considered necessary peptides were purified on a preparative scale. The correct identities of the peptides were confirmed by matrix assisted laser desorption ionisation mass spectroscopy or electrospray ionization mass spectrometry. The purity was checked by reverse phase HPLC.

The “multimeric” peptides may be synthesised by standardised peptide synthesis methodologies using an orthogonal protection strategy (e.g., as described by Cwirla et al. Science, Vol 276, 1997, pp 1696-1699). Alternatively, heterogeneous methods well known to peptide chemists for construction of e.g. multimeric antigens for immunisation (D. N. Posnett et al (1988) J. Biol. Chem. 263:1719-1725) are also applicable within the present invention.

In one embodiment of the first aspect of the present invention, the uPAR binding peptide is a monomeric peptide or multimeric peptide wherein the peptide constituent(s) is/are selected from the group consisting of:

(D-Asp)-([beta]-cyclohexyl-L-alanine)-(Phe)-(D-Ser)-(D-Arg)-(Tyr)-
(Leu)-(Trp)-(Ser),
(Ser)-(Leu)-([beta]-cyclohexyl-L-alanine)-(Phe)-(D-Ser)-(Gln)-
(Tyr)(Leu)-(Trp)-(Ser),
(D-Glu)-([beta]-cyclohexyl-L-alanine)-(Phe)-(D-Ser)-(D-Tyr)-(Tyr)-
(Leu)-(Trp)-(Ser),
(Asp)-([beta]-cyclohexyl-L-alanine)-(Phe)-(D-Ser)-(D-Arg)-(Tyr)-
(Leu)-(Trp)-(Ser),
(Asp)-([beta]-cyclohexyl-L-alanine)-(Phe)-(Ser)-(D-Arg)-(Tyr)-Leu)-
(Trp)-(Ser),
(D-Asp)-([beta]-cyclohexyl-L-alanine)-(Phe)-(Ser)-(D-Arg)-(Tyr)-Leu)-
(Trp)-(Ser),
(D-Thr)-([beta]-cyclohexyl-L-alanine)-(Phe)-(D-Ser)-(D-Arg)-(Tyr)-
(Leu)-(Trp)-(Ser),
(D-Asp)-([beta]-cyclohexyl-L-alanine)-(Phe)-(D-Ser)-(D-Arg)-(Tyr)-
(Leu)-([beta]-2-naphthyl-L-alanine)-(Ser),
(Asp)-([beta]-cyclohexyl-L-alanine)-(Phe)-(D-Ser)-(Arg)-(Tyr)-(Leu)-
(Trp)-(Ser),
(Asp)-([beta]-cyclohexyl-L-alanine)-(Phe)-(D-Ser)-(D-Arg)-(Tyr)-
(Leu)([beta]-1-naphthyl-L-alanine)-(Ser),
(D-Glu)-([beta]-cyclohexyl-L-alanine)-(Phe)-(D-Ser)-(Tyr)-(Tyr)-
(Leu)-(Trp)-(Ser),
(Asp)-([beta]-cyclohexyl-L-alanine)-(Phe)-(D-Ser)-(D-Arg)-(Leu)-
(Leu)-(Trp)-(D-His),
(Asp)-([beta]-cyclohexyl-L-alanine)-(Phe)-(D-Ser)-(D-Arg)-([beta]-
cyclohexyl-L-alanine)-(Leu)-(Trp)-(Ile),
(Asp)-([beta]-cyclohexyl-L-alanine)-(Phe)-(D-Ser)-(D-Arg)-(Tyr)-
(Leu)([beta]-1-naphthyl-L-alanine)-(D-His),
(Asp)-([beta]-cyclohexyl-L-alanine)-(Phe)-(D-Ser)-(D-Arg)-(N-(2,3-
dimethoxybenzyl)glycine)-(D-Phe)-(N-(3-indolylethyl)glycine)-(N-
(2-methoxyethyl)glycine),
(Asp)-([beta]-cyclohexyl-L-alanine)-(Phe)-(D-Ser)-(D-Arg)-(N-(2,3-
dimethoxybenzyl)glycine)-(D-Phe)-(N-benzylglycine)-(N-
(2[beta]thoxyethyl)glycine),
(Asp)-([beta]-cyclohexyl-L-alanine)-(Phe)-(D-Ser)-(D-Arg)-(N-(2,3-
dimethoxybenzyl)glycine)-(D-Phe)-(N-(methylnaphthalyl)glycine)-(N-
(2-methoxyethyl)glycine), and
(Asp)-([beta]-cyclohexyl-L-alanine)-(Phe)-(D-Ser)-(D-Arg)-(N-(2,3-
dimethoxybenzyl)glycine)-(D-Phe)-(N-(2,3-dimethoxybenzyl)glycine)-
(Ile).

In a preferred embodiment of the first aspect of the present invention, the uPAR binding peptide is a monomeric peptide selected from the group consisting of:

(D-Asp)-([beta]-cyclohexyl-L-alanine)-(Phe)-(D-Ser)-(D-Arg)-(Tyr)-(Leu)-(Trp)-(Ser),
(Ser)-(Leu)-([beta]-cyclohexyl-L-alanine)-(Phe)-(D-Ser)-(Gln)-(Tyr)(Leu)-(Trp)-(Ser),
(D-Glu)-([beta]-cyclohexyl-L-alanine)-(Phe)-(D-Ser)-(D-Tyr)-(Tyr)-(Leu)-(Trp)-(Ser),
(Asp)-([beta]-cyclohexyl-L-alanine)-(Phe)-(D-Ser)-(D-Arg)-(Tyr)-(Leu)-(Trp)-(Ser),
(Asp)-([beta]-cyclohexyl-L-alanine)-(Phe)-(Ser)-(D-Arg)-(Tyr)-Leu)-(Trp)-(Ser),
(D-Asp)-([beta]-cyclohexyl-L-alanine)-(Phe)-(Ser)-(D-Arg)-(Tyr)-Leu)-(Trp)-(Ser),
(D-Thr)-([beta]-cyclohexyl-L-alanine)-(Phe)-(D-Ser)-(D-Arg)-(Tyr)-(Leu)-(Trp)-(Ser),
(D-Asp)-([beta]-cyclohexyl-L-alanine)-(Phe)-(D-Ser)-(D-Arg)-(Tyr)-(Leu)-([beta]-2-
naphthyl-L-alanine)-(Ser),
(Asp)-([beta]-cyclohexyl-L-alanine)-(Phe)-(D-Ser)-(Arg)-(Tyr)-(Leu)-(Trp)-(Ser),
(Asp)-([beta]-cyclohexyl-L-alanine)-(Phe)-(D-Ser)-(D-Arg)-(Tyr)-(Leu)([beta]-1-
naphthyl-L-alanine)-(Ser),
(D-Glu)-([beta]-cyclohexyl-L-alanine)-(Phe)-(D-Ser)-(Tyr)-(Tyr)-(Leu)-(Trp)-(Ser),
(Asp)-([beta]-cyclohexyl-L-alanine)-(Phe)-(D-Ser)-(D-Arg)-(Leu)-(Leu)-(Trp)-(D-His),
(Asp)-([beta]-cyclohexyl-L-alanine)-(Phe)-(D-Ser)-(D-Arg)-([beta]-cyclohexyl-L-
alanine)-(Leu)-(Trp)-(Ile),
(Asp)-([beta]-cyclohexyl-L-alanine)-(Phe)-(D-Ser)-(D-Arg)-(Tyr)-(Leu)([beta]-1-
naphthyl-L-alanine)-(D-His),
(Asp)-([beta]-cyclohexyl-L-alanine)-(Phe)-(D-Ser)-(D-Arg)-(N-(2,3-
dimethoxybenzyl)glycine)-(D-Phe)-(N-(3-indolylethyl)glycine)-(N-(2-methoxyethyl)
glycine),
(Asp)-([beta]-cyclohexyl-L-alanine)-(Phe)-(D-Ser)-(D-Arg)-(N-(2,3-
dimethoxybenzyl)glycine)-(D-Phe)-(N-benzylglycine)-(N-(2[beta]thoxyethyl)glycine),
(Asp)-([beta]-cyclohexyl-L-alanine)-(Phe)-(D-Ser)-(D-Arg)-(N-(2,3-
dimethoxybenzyl)glycine)-(D-Phe)-(N-(methylnaphthalyl)glycine)-(N-(2-
methoxyethyl)glycine), and
(Asp)-([beta]-cyclohexyl-L-alanine)-(Phe)-(D-Ser)-(D-Arg)-(N-(2,3-
dimethoxybenzyl)glycine)-(D-Phe)-(N-(2,3-dimethoxybenzyl)glycine)-Ile).

These peptides all share a minimum structure by having at least part of the binding hotspot of x-Cha-Phe-x-x-x-Leu-Trp-x, where the residues in bold are hot spots for interaction with uPAR, Cha is Cyclohexyl-(L) alanine, and x designate any amino acid. These peptides in themselves, have high specificity towards the target uPAR as well as good affinity thereby facilitating sufficient retention of the resulting radionuclide labelled uPAR binding peptide conjugates at the target for the radionuclide to have sufficient time to provide a therapeutic relevant radioactive exposure of the targeted area. Furthermore, the peptides provide good compatibility with suitable chelating agents including DOTA, DOTAM, NOTA, NODAGA, SARCOPHAGINE, CB-TE2A and derivatives thereof, in particular DOTA.

In a most preferred embodiment of the first aspect of the present invention, the uPAR binding peptide is (D-Asp)-([beta]-cyclohexyl-L-alanine)-(Phe)-(D-Ser)-(D-Arg)-(Tyr)-(Leu)-(Trp)-(Ser) also designated AE105 in the context of the present invention. Thereby providing a uPAR binding peptide which in itself has superior specificity towards the target uPAR as well as high affinity thereby facilitating superior retention of the radionuclide labelled uPAR binding peptide conjugates at the target for the radionuclide to have sufficient time to provide a therapeutic relevant radioactive exposure of the targeted area. Furthermore, the uPAR binding peptide provides good compatibility with suitable chelating agents including DOTA, DOTAM, NOTA, NODAGA, SARCOPHAGINE, CB-TE2A and derivatives thereof, in particular DOTA and superior target binding capabilities when labelled with the selected radionuclides 67Cu, 225Ac, 212Pb, 161Tb, and 149Tb, in particular 67Cu, 212Pb, and 225Ac.

Radiolabeling of the peptide(s) selected for the radionuclide labelled uPAR binding peptide conjugates of the present invention is enabled by coupling a chelating agent suitable for binding radiometals to the peptide. The chelating agent is capable of binding a selected radionuclide thereto.

### Chelator

The use of various chelating agents suitable for binding radiometals for radio labeling peptides is well known in the art. Suitable chelating agents generally include those which contain a tetradentate ligand with at least one sulfur group available for binding the metal radionuclide such as the known N3S and N2S2 ligands. The chelating agent is coupled to the uPAR binding peptide by standard methodology known in the field of the invention and may be added at any location on the uPAR binding peptide, provided that the biological activity such as binding properties and specificity of the peptide is not adversely affected. Preferably, the chelating group is covalently coupled to the amino terminal amino acid of the peptide. The chelating group may advantageously be attached to the peptide during solid phase peptide synthesis or added by solution phase chemistry after the peptide has been obtained. Preferred chelating agents include DOTA (1,4,7,10-tetrakis(carboxymethyl)-1,4,7,10 tetraazacyclo dodecane), DOTAM (2-[4,7,10-tris(2-amino-2-oxoethyl)-1,4,7,10-tetrazacyclododec-1-yl]acetamide), NOTA (2,2′,2″-(1,4,7-triazacyclononane-1,4,7-triyl)triacetic acid), NODAGA, SARCOPHAGINE (3,6,10,13,16,19-hexaazabicyclo(6,6,6) icosane), CB-TE2A (1,4,8,11-Tetraazabicyclo[6.6.2]hexadecane-4,11-diacetic acid) and derivatives thereof, which constitute an important class of chelators for biomedical applications as they accommodate very stably a variety of di- and trivalent metal ions.

DOTA (Dodecane tetraacetic acid) with the chemical formula (1,4,7,10-tetrakis(carboxymethyl)-1,4,7,10 tetraazacyclo dodecane) and its derivatives, are particularly acknowledged to constitute an important class of chelators for biomedical applications as they accommodate very stably a variety of di- and trivalent metal ions, such as 67Cu, 225Ac, 212Pb, 161Tb and 149Tb.

DOTAM also known as TCMC is a DOTA analogue.

SARCOPHAGINE (Sar) is a bicyclic cage-like chelator molecule derived from cyclam. The chemical formula of sarcophagine is 3,6,10,13,16,19-hexaazabicyclo(6,6,6) icosane and additional functional or non-functional groups are often linked to this structure, thereby creating derivatives of sarcophagine, such as in DiAmSar (1,8-diamino-Sar), AmBaSar (4-((8-amino-3,6,10,13,16,19-hexaazabicyclo[6.6.6] icosane-1-ylamino)methyl)benzoic acid), and MeCOSar (5-(8-methyl-3,6, 10, 13, 16, 19-hexaaza-bicyclo[6.6.6]icosan-1-ylamino)-5-oxopentanoic acid). Sarcophagine and derivatives thereof are suitable chelating agents for use in the radionuclide labelled uPAR binding peptide conjugates of the present invention and are preferred for 67Cu labelled uPAR binding peptide conjugates of the present invention.

In one embodiment of the first aspect of the present invention the chelating agent is selected from the group consisting of DOTA, DOTAM, NOTA, NODAGA, SARCOPHAGINE, CB-TE2A and derivatives thereof, preferably DOTA, DOTAM, NOTA, NODAGA, SARCOPHAGINE, DiAmSar, AmBaSar, MeCOSar, or CB-TE2A.

In a preferred embodiment of the first aspect of the present invention the chelating agent is selected from the group consisting of DOTA, DOTAM, SARCOPHAGINE, and derivatives thereof, preferably DOTA, DOTAM, SARCOPHAGINE, DiAmSar, AmBaSar, or MeCOSar.

In a more preferred embodiment of the first aspect of the present invention the chelating agent is DOTA.

In one embodiment of the first aspect of the present invention the chelating agent of the uPAR binding peptide conjugate is DOTA, and the radionuclide is selected from the group consisting of 67Cu, 225Ac, 212Pb, 161Tb, and 149Tb, preferably 67Cu, 225Ac, or 212Pb, more preferably 67Cu or 225Ac, most preferred 67Cu.

In one embodiment of the first aspect of the present invention the chelating agent is DOTA and the uPAR binding peptide is selected from the group consisting of (D-Asp)-([beta]-cyclohexyl-L-alanine)-(Phe)-(D-Ser)-(D-Arg)-(Tyr)-(Leu)-(Trp)-(Ser), (Ser)-(Leu)-([beta]-cyclohexyl-L-alanine)-(Phe)-(D-Ser)-(Gln)-(Tyr) (Leu)-(Trp)-(Ser), (D-Glu)-([beta]-cyclohexyl-L-alanine)-(Phe)-(D-Ser)-(D-Tyr)-(Tyr)-(Leu)-(Trp)-(Ser), (Asp)-([beta]-cyclohexyl-L-alanine)-(Phe)-(D-Ser)-(D-Arg)-(Tyr)-(Leu)-(Trp)-(Ser), (Asp)-([beta]-cyclohexyl-L-alanine)-(Phe)-(Ser)-(D-Arg)-(Tyr)-Leu)-(Trp)-(Ser), (D-Asp)-([beta]-cyclohexyl-L-alanine)-(Phe)-(Ser)-(D-Arg)-(Tyr)-Leu)-(Trp)-(Ser), (D-Thr)-([beta]-cyclohexyl-L-alanine)-(Phe)-(D-Ser)-(D-Arg)-(Tyr)-(Leu)-(Trp)-(Ser), (D-Asp)-([beta]-cyclohexyl-L-alanine)-(Phe)-(D-Ser)-(D-Arg)-(Tyr)-(Leu)-([beta]-2-naphthyl-L-alanine)-(Ser), (Asp)-([beta]-cyclohexyl-L-alanine)-(Phe)-(D-Ser)-(Arg)-(Tyr)-(Leu)-(Trp)-(Ser), (Asp)-([beta]-cyclohexyl-L-alanine)-(Phe)-(D-Ser)-(D-Arg)-(Tyr)-(Leu) ([beta]-1-naphthyl-L-alanine)-(Ser), (D-Glu)-([beta]-cyclohexyl-L-alanine)-(Phe)-(D-Ser)-(Tyr)-(Tyr)-(Leu)-(Trp)-(Ser), (Asp)-([beta]-cyclohexyl-L-alanine)-(Phe)-(D-Ser)-(D-Arg)-(Leu)-(Leu)-(Trp)-(D-His), (Asp)-([beta]-cyclohexyl-L-alanine)-(Phe)-(D-Ser)-(D-Arg)-([beta]-cyclohexyl-L-alanine)-(Leu)-(Trp)-(Ile), (Asp)-([beta]-cyclohexyl-L-alanine)-(Phe)-(D-Ser)-(D-Arg)-(Tyr)-(Leu) ([beta]-1-naphthyl-L-alanine)-(D-His), (Asp)-([beta]-cyclohexyl-L-alanine)-(Phe)-(D-Ser)-(D-Arg)-(N-(2,3-dimethoxybenzyl)glycine)-(D-Phe)-(N-(3-indolylethyl)glycine)-(N-(2-methoxyethyl)glycine), (Asp)-([beta]-cyclohexyl-L-alanine)-(Phe)-(D-Ser)-(D-Arg)-(N-(2,3-dimethoxybenzyl)glycine)-(D-Phe)-(N-benzylglycine)-(N-(2 [beta]thoxyethyl)glycine), (Asp)-([beta]-cyclohexyl-L-alanine)-(Phe)-(D-Ser)-(D-Arg)-(N-(2,3-dimethoxybenzyl)glycine)-(D-Phe)-(N-(methylnaphthalyl)glycine)-(N-(2-methoxyethyl)glycine), and (Asp)-([beta]-cyclohexyl-L-alanine)-(Phe)-(D-Ser)-(D-Arg)-(N-(2,3-dimethoxybenzyl)glycine)-(D-Phe)-(N-(2,3-dimethoxybenzyl)glycine)-(Ile), and the radionuclide is selected from the group consisting of 67Cu, 225Ac, 212Pb, 161Tb, and 149Tb, preferably 67Cu, 225Ac, or 212Pb, more preferably 67Cu or 225Ac, most preferred 67Cu.

In a preferred embodiment of the first aspect of the present invention the chelating agent is DOTA and the uPAR binding peptide is (D-Asp)-([beta]-cyclohexyl-L-alanine)-(Phe)-(D-Ser)-(D-Arg)-(Tyr)-(Leu)-(Trp)-(Ser) and the radionuclide is selected from the group consisting of 67Cu, 225Ac, 212Pb, 161Tb, and 149Tb, preferably 67Cu, 225Ac, or 212Pb, more preferably 67Cu or 225Ac, most preferred 67Cu.

In one embodiment of the first aspect of the present invention the chelating agent of the uPAR binding peptide conjugate is DOTAM and the radionuclide is selected from the group consisting of 67Cu, 225Ac, 212Pb, 161Tb, and 149Tb, preferably 67Cu, 225Ac, or 212Pb, more preferably 67Cu or 225Ac, most preferred 67Cu.

In one embodiment of the first aspect of the present invention the chelating agent is DOTAM and the uPAR binding peptide is selected from the group consisting of (D-Asp)-([beta]-cyclohexyl-L-alanine)-(Phe)-(D-Ser)-(D-Arg)-(Tyr)-(Leu)-(Trp)-(Ser), (Ser)-(Leu)-([beta]-cyclohexyl-L-alanine)-(Phe)-(D-Ser)-(Gln)-(Tyr) (Leu)-(Trp)-(Ser), (D-Glu)-([beta]-cyclohexyl-L-alanine)-(Phe)-(D-Ser)-(D-Tyr)-(Tyr)-(Leu)-(Trp)-(Ser), (Asp)-([beta]-cyclohexyl-L-alanine)-(Phe)-(D-Ser)-(D-Arg)-(Tyr)-(Leu)-(Trp)-(Ser), (Asp)-([beta]-cyclohexyl-L-alanine)-(Phe)-(Ser)-(D-Arg)-(Tyr)-Leu)-(Trp)-(Ser), (D-Asp)-([beta]-cyclohexyl-L-alanine)-(Phe)-(Ser)-(D-Arg)-(Tyr)-Leu)-(Trp)-(Ser), (D-Thr)-([beta]-cyclohexyl-L-alanine)-(Phe)-(D-Ser)-(D-Arg)-(Tyr)-(Leu)-(Trp)-(Ser), (D-Asp)-([beta]-cyclohexyl-L-alanine)-(Phe)-(D-Ser)-(D-Arg)-(Tyr)-(Leu)-([beta]-2-naphthyl-L-alanine)-(Ser), (Asp)-([beta]-cyclohexyl-L-alanine)-(Phe)-(D-Ser)-(Arg)-(Tyr)-(Leu)-(Trp)-(Ser), (Asp)-([beta]-cyclohexyl-L-alanine)-(Phe)-(D-Ser)-(D-Arg)-(Tyr)-(Leu) ([beta]-1-naphthyl-L-alanine)-(Ser), (D-Glu)-([beta]-cyclohexyl-L-alanine)-(Phe)-(D-Ser)-(Tyr)-(Tyr)-(Leu)-(Trp)-(Ser), (Asp)-([beta]-cyclohexyl-L-alanine)-(Phe)-(D-Ser)-(D-Arg)-(Leu)-(Leu)-(Trp)-(D-His), (Asp)-([beta]-cyclohexyl-L-alanine)-(Phe)-(D-Ser)-(D-Arg)-([beta]-cyclohexyl-L-alanine)-(Leu)-(Trp)-(Ile), (Asp)-([beta]-cyclohexyl-L-alanine)-(Phe)-(D-Ser)-(D-Arg)-(Tyr)-(Leu) ([beta]-1-naphthyl-L-alanine)-(D-His), (Asp)-([beta]-cyclohexyl-L-alanine)-(Phe)-(D-Ser)-(D-Arg)-(N-(2,3-dimethoxybenzyl)glycine)-(D-Phe)-(N-(3-indolylethyl)glycine)-(N-(2-methoxyethyl)glycine), (Asp)-([beta]-cyclohexyl-L-alanine)-(Phe)-(D-Ser)-(D-Arg)-(N-(2,3-dimethoxybenzyl)glycine)-(D-Phe)-(N-benzylglycine)-(N-(2 [beta]thoxyethyl)glycine), (Asp)-([beta]-cyclohexyl-L-alanine)-(Phe)-(D-Ser)-(D-Arg)-(N-(2,3-dimethoxybenzyl)glycine)-(D-Phe)-(N-(methylnaphthalyl)glycine)-(N-(2-methoxyethyl)glycine), and (Asp)-([beta]-cyclohexyl-L-alanine)-(Phe)-(D-Ser)-(D-Arg)-(N-(2,3-dimethoxybenzyl)glycine)-(D-Phe)-(N-(2,3-dimethoxybenzyl)glycine)-(Ile), And the radionuclide is selected from the group consisting of 67Cu, 225Ac, 212Pb, 161Tb, and 149Tb, preferably 67Cu, 225Ac, or 212Pb, more preferably 67Cu or 225Ac, most preferred 67Cu.

In a preferred embodiment of the first aspect of the present invention the chelating agent is DOTAM and the uPAR binding peptide is (D-Asp)-([beta]-cyclohexyl-L-alanine)-(Phe)-(D-Ser)-(D-Arg)-(Tyr)-(Leu)-(Trp)-(Ser) and the radionuclide is selected from the group consisting of 67Cu, 225Ac, 212Pb, 161Tb and 149Tb, preferably 67Cu, 225Ac or 212Pb, more preferably 67Cu or 225Ac, most preferred 67Cu.

In another preferred embodiment of the first aspect of the present invention the chelating agent is DOTAM and the uPAR binding peptide is (D-Asp)-([beta]-cyclohexyl-L-alanine)-(Phe)-(D-Ser)-(D-Arg)-(Tyr)-(Leu)-(Trp)-(Ser) and the radionuclide is 212Pb.

In one embodiment of the first aspect of the present invention the chelating agent of the uPAR binding peptide conjugate is NOTA, and the radionuclide is selected from the group consisting of 67Cu, 225Ac, 212Pb, 161Tb, and 149Tb, preferably 67Cu, 225Ac, or 212Pb, more preferably 67Cu or 225Ac, most preferred 67Cu.

In one embodiment of the first aspect of the present invention the chelating agent is NOTA and the uPAR binding peptide is selected from the group consisting of (D-Asp)-([beta]-cyclohexyl-L-alanine)-(Phe)-(D-Ser)-(D-Arg)-(Tyr)-(Leu)-(Trp)-(Ser), (Ser)-(Leu)-([beta]-cyclohexyl-L-alanine)-(Phe)-(D-Ser)-(Gln)-(Tyr) (Leu)-(Trp)-(Ser), (D-Glu)-([beta]-cyclohexyl-L-alanine)-(Phe)-(D-Ser)-(D-Tyr)-(Tyr)-(Leu)-(Trp)-(Ser), (Asp)-([beta]-cyclohexyl-L-alanine)-(Phe)-(D-Ser)-(D-Arg)-(Tyr)-(Leu)-(Trp)-(Ser), (Asp)-([beta]-cyclohexyl-L-alanine)-(Phe)-(Ser)-(D-Arg)-(Tyr)-Leu)-(Trp)-(Ser), (D-Asp)-([beta]-cyclohexyl-L-alanine)-(Phe)-(Ser)-(D-Arg)-(Tyr)-Leu)-(Trp)-(Ser), (D-Thr)-([beta]-cyclohexyl-L-alanine)-(Phe)-(D-Ser)-(D-Arg)-(Tyr)-(Leu)-(Trp)-(Ser), (D-Asp)-([beta]-cyclohexyl-L-alanine)-(Phe)-(D-Ser)-(D-Arg)-(Tyr)-(Leu)-([beta]-2-naphthyl-L-alanine)-(Ser), (Asp)-([beta]-cyclohexyl-L-alanine)-(Phe)-(D-Ser)-(Arg)-(Tyr)-(Leu)-(Trp)-(Ser), (Asp)-([beta]-cyclohexyl-L-alanine)-(Phe)-(D-Ser)-(D-Arg)-(Tyr)-(Leu) ([beta]-1-naphthyl-L-alanine)-(Ser), (D-Glu)-([beta]-cyclohexyl-L-alanine)-(Phe)-(D-Ser)-(Tyr)-(Tyr)-(Leu)-(Trp)-(Ser), (Asp)-([beta]-cyclohexyl-L-alanine)-(Phe)-(D-Ser)-(D-Arg)-(Leu)-(Leu)-(Trp)-(D-His), (Asp)-([beta]-cyclohexyl-L-alanine)-(Phe)-(D-Ser)-(D-Arg)-([beta]-cyclohexyl-L-alanine)-(Leu)-(Trp)-(Ile), (Asp)-([beta]-cyclohexyl-L-alanine)-(Phe)-(D-Ser)-(D-Arg)-(Tyr)-(Leu) ([beta]-1-naphthyl-L-alanine)-(D-His), (Asp)-([beta]-cyclohexyl-L-alanine)-(Phe)-(D-Ser)-(D-Arg)-(N-(2,3-dimethoxybenzyl)glycine)-(D-Phe)-(N-(3-indolylethyl)glycine)-(N-(2-methoxyethyl)glycine), (Asp)-([beta]-cyclohexyl-L-alanine)-(Phe)-(D-Ser)-(D-Arg)-(N-(2,3-dimethoxybenzyl)glycine)-(D-Phe)-(N-benzylglycine)-(N-(2 [beta]thoxyethyl)glycine), (Asp)-([beta]-cyclohexyl-L-alanine)-(Phe)-(D-Ser)-(D-Arg)-(N-(2,3-dimethoxybenzyl)glycine)-(D-Phe)-(N-(methylnaphthalyl)glycine)-(N-(2-methoxyethyl)glycine), and (Asp)-([beta]-cyclohexyl-L-alanine)-(Phe)-(D-Ser)-(D-Arg)-(N-(2,3-dimethoxybenzyl)glycine)-(D-Phe)-(N-(2,3-dimethoxybenzyl)glycine)-(Ile), And the radionuclide is selected from the group consisting of 67Cu, 225Ac, 212Pb, 161Tb, and 149Tb, preferably 67Cu, 225Ac, or 212Pb, more preferably 67Cu or 225Ac, most preferred 67Cu.

In a preferred embodiment of the first aspect of the present invention the chelating agent is NOTA and the uPAR binding peptide is (D-Asp)-([beta]-cyclohexyl-L-alanine)-(Phe)-(D-Ser)-(D-Arg)-(Tyr)-(Leu)-(Trp)-(Ser), and the radionuclide is selected from the group consisting of 67Cu, 225Ac, 212Pb, 161Tb, and 149Tb, preferably 67Cu, 225Ac, or 212Pb, more preferably 67Cu or 225Ac, most preferred 67Cu.

In one embodiment of the first aspect of the present invention the chelating agent of the uPAR binding peptide conjugate is NODAGA and the radionuclide is selected from the group consisting of 67Cu, 225Ac, 212Pb, 161Tb, and 149Tb, preferably 67Cu, 225Ac, or 212Pb, more preferably 67Cu or 225Ac, most preferred 67Cu.

In one embodiment of the first aspect of the present invention the chelating agent is NODAGA and the uPAR binding peptide is selected from the group consisting of (D-Asp)-([beta]-cyclohexyl-L-alanine)-(Phe)-(D-Ser)-(D-Arg)-(Tyr)-(Leu)-(Trp)-(Ser), (Ser)-(Leu)-([beta]-cyclohexyl-L-alanine)-(Phe)-(D-Ser)-(Gln)-(Tyr) (Leu)-(Trp)-(Ser), (D-Glu)-([beta]-cyclohexyl-L-alanine)-(Phe)-(D-Ser)-(D-Tyr)-(Tyr)-(Leu)-(Trp)-(Ser), (Asp)-([beta]-cyclohexyl-L-alanine)-(Phe)-(D-Ser)-(D-Arg)-(Tyr)-(Leu)-(Trp)-(Ser), (Asp)-([beta]-cyclohexyl-L-alanine)-(Phe)-(Ser)-(D-Arg)-(Tyr)-Leu)-(Trp)-(Ser), (D-Asp)-([beta]-cyclohexyl-L-alanine)-(Phe)-(Ser)-(D-Arg)-(Tyr)-Leu)-(Trp)-(Ser), (D-Thr)-([beta]-cyclohexyl-L-alanine)-(Phe)-(D-Ser)-(D-Arg)-(Tyr)-(Leu)-(Trp)-(Ser), (D-Asp)-([beta]-cyclohexyl-L-alanine)-(Phe)-(D-Ser)-(D-Arg)-(Tyr)-(Leu)-([beta]-2-naphthyl-L-alanine)-(Ser), (Asp)-([beta]-cyclohexyl-L-alanine)-(Phe)-(D-Ser)-(Arg)-(Tyr)-(Leu)-(Trp)-(Ser), (Asp)-([beta]-cyclohexyl-L-alanine)-(Phe)-(D-Ser)-(D-Arg)-(Tyr)-(Leu) ([beta]-1-naphthyl-L-alanine)-(Ser), (D-Glu)-([beta]-cyclohexyl-L-alanine)-(Phe)-(D-Ser)-(Tyr)-(Tyr)-(Leu)-(Trp)-(Ser), (Asp)-([beta]-cyclohexyl-L-alanine)-(Phe)-(D-Ser)-(D-Arg)-(Leu)-(Leu)-(Trp)-(D-His), (Asp)-([beta]-cyclohexyl-L-alanine)-(Phe)-(D-Ser)-(D-Arg)-([beta]-cyclohexyl-L-alanine)-(Leu)-(Trp)-(Ile), (Asp)-([beta]-cyclohexyl-L-alanine)-(Phe)-(D-Ser)-(D-Arg)-(Tyr)-(Leu) ([beta]-1-naphthyl-L-alanine)-(D-His), (Asp)-([beta]-cyclohexyl-L-alanine)-(Phe)-(D-Ser)-(D-Arg)-(N-(2,3-dimethoxybenzyl)glycine)-(D-Phe)-(N-(3-indolylethyl)glycine)-(N-(2-methoxyethyl)glycine), (Asp)-([beta]-cyclohexyl-L-alanine)-(Phe)-(D-Ser)-(D-Arg)-(N-(2,3-dimethoxybenzyl)glycine)-(D-Phe)-(N-benzylglycine)-(N-(2 [beta]thoxyethyl)glycine), (Asp)-([beta]-cyclohexyl-L-alanine)-(Phe)-(D-Ser)-(D-Arg)-(N-(2,3-dimethoxybenzyl)glycine)-(D-Phe)-(N-(methylnaphthalyl)glycine)-(N-(2-methoxyethyl)glycine), and (Asp)-([beta]-cyclohexyl-L-alanine)-(Phe)-(D-Ser)-(D-Arg)-(N-(2,3-dimethoxybenzyl)glycine)-(D-Phe)-(N-(2,3-dimethoxybenzyl)glycine)-(Ile), and the radionuclide is selected from the group consisting of 67Cu, 225Ac, 212Pb, 161Tb, and 149Tb, preferably 67Cu, 225Ac, or 212Pb, more preferably 67Cu or 225Ac, most preferred 67Cu.

In a preferred embodiment of the first aspect of the present invention the chelating agent is NODAGA and the uPAR binding peptide is (D-Asp)-([beta]-cyclohexyl-L-alanine)-(Phe)-(D-Ser)-(D-Arg)-(Tyr)-(Leu)-(Trp)-(Ser), and the radionuclide is selected from the group consisting of 67Cu, 225Ac, 212Pb, 161Tb, and 149Tb, preferably 67Cu, 225Ac, or 212Pb, more preferably 67Cu or 225Ac, most preferred 67Cu.

In one embodiment of the first aspect of the present invention the chelating agent of the uPAR binding peptide conjugate is MeCOSar and the radionuclide is selected from the group consisting of 67Cu, 225Ac, 212Pb, 161Tb, and 149Tb, preferably 67Cu, 225Ac, or 212Pb, more preferably 67Cu or 225Ac, most preferred 67Cu.

In one embodiment of the first aspect of the present invention the chelating agent is MeCOSar and the uPAR binding peptide is selected from the group consisting of (D-Asp)-([beta]-cyclohexyl-L-alanine)-(Phe)-(D-Ser)-(D-Arg)-(Tyr)-(Leu)-(Trp)-(Ser), (Ser)-(Leu)-([beta]-cyclohexyl-L-alanine)-(Phe)-(D-Ser)-(Gln)-(Tyr) (Leu)-(Trp)-(Ser), (D-Glu)-([beta]-cyclohexyl-L-alanine)-(Phe)-(D-Ser)-(D-Tyr)-(Tyr)-(Leu)-(Trp)-(Ser), (Asp)-([beta]-cyclohexyl-L-alanine)-(Phe)-(D-Ser)-(D-Arg)-(Tyr)-(Leu)-(Trp)-(Ser), (Asp)-([beta]-cyclohexyl-L-alanine)-(Phe)-(Ser)-(D-Arg)-(Tyr)-Leu)-(Trp)-(Ser), (D-Asp)-([beta]-cyclohexyl-L-alanine)-(Phe)-(Ser)-(D-Arg)-(Tyr)-Leu)-(Trp)-(Ser), (D-Thr)-([beta]-cyclohexyl-L-alanine)-(Phe)-(D-Ser)-(D-Arg)-(Tyr)-(Leu)-(Trp)-(Ser), (D-Asp)-([beta]-cyclohexyl-L-alanine)-(Phe)-(D-Ser)-(D-Arg)-(Tyr)-(Leu)-([beta]-2-naphthyl-L-alanine)-(Ser), (Asp)-([beta]-cyclohexyl-L-alanine)-(Phe)-(D-Ser)-(Arg)-(Tyr)-(Leu)-(Trp)-(Ser), (Asp)-([beta]-cyclohexyl-L-alanine)-(Phe)-(D-Ser)-(D-Arg)-(Tyr)-(Leu) ([beta]-1-naphthyl-L-alanine)-(Ser), (D-Glu)-([beta]-cyclohexyl-L-alanine)-(Phe)-(D-Ser)-(Tyr)-(Tyr)-(Leu)-(Trp)-(Ser), (Asp)-([beta]-cyclohexyl-L-alanine)-(Phe)-(D-Ser)-(D-Arg)-(Leu)-(Leu)-(Trp)-(D-His), (Asp)-([beta]-cyclohexyl-L-alanine)-(Phe)-(D-Ser)-(D-Arg)-([beta]-cyclohexyl-L-alanine)-(Leu)-(Trp)-(Ile), (Asp)-([beta]-cyclohexyl-L-alanine)-(Phe)-(D-Ser)-(D-Arg)-(Tyr)-(Leu) ([beta]-1-naphthyl-L-alanine)-(D-His), (Asp)-([beta]-cyclohexyl-L-alanine)-(Phe)-(D-Ser)-(D-Arg)-(N-(2,3-dimethoxybenzyl)glycine)-(D-Phe)-(N-(3-indolylethyl)glycine)-(N-(2-methoxyethyl)glycine), (Asp)-([beta]-cyclohexyl-L-alanine)-(Phe)-(D-Ser)-(D-Arg)-(N-(2,3-dimethoxybenzyl)glycine)-(D-Phe)-(N-benzylglycine)-(N-(2 [beta]thoxyethyl)glycine), (Asp)-([beta]-cyclohexyl-L-alanine)-(Phe)-(D-Ser)-(D-Arg)-(N-(2,3-dimethoxybenzyl)glycine)-(D-Phe)-(N-(methylnaphthalyl)glycine)-(N-(2-methoxyethyl)glycine), and (Asp)-([beta]-cyclohexyl-L-alanine)-(Phe)-(D-Ser)-(D-Arg)-(N-(2,3-dimethoxybenzyl)glycine)-(D-Phe)-(N-(2,3-dimethoxybenzyl)glycine)-(Ile), and the radionuclide is selected from the group consisting of 67Cu, 225Ac, 212Pb, 161Tb, and 149Tb, preferably 67Cu, 225Ac, or 212Pb, more preferably 67Cu or 225Ac, most preferred 67Cu.

In a preferred embodiment of the first aspect of the present invention the chelating agent is MeCOSar and the uPAR binding peptide is (D-Asp)-([beta]-cyclohexyl-L-alanine)-(Phe)-(D-Ser)-(D-Arg)-(Tyr)-(Leu)-(Trp)-(Ser), and the radionuclide is selected from the group consisting of 67Cu, 225Ac, 212Pb, 161Tb, and 149Tb, preferably 67Cu, 225Ac, or 212Pb, more preferably 67Cu or 225Ac, most preferred 67Cu.

In one embodiment of the first aspect of the present invention the chelating agent of the uPAR binding peptide conjugate is CB-TE2A, and the radionuclide is selected from the group consisting of 67Cu, 225Ac, 212Pb, 161Tb, and 149Tb, preferably 67Cu, 225Ac, or 212Pb, more preferably 67Cu or 225Ac, most preferred 67Cu.

In one embodiment of the first aspect of the present invention the chelating agent is CB-TE2A and the uPAR binding peptide is selected from the group consisting of (D-Asp)-([beta]-cyclohexyl-L-alanine)-(Phe)-(D-Ser)-(D-Arg)-(Tyr)-(Leu)-(Trp)-(Ser), (Ser)-(Leu)-([beta]-cyclohexyl-L-alanine)-(Phe)-(D-Ser)-(Gln)-(Tyr) (Leu)-(Trp)-(Ser), (D-Glu)-([beta]-cyclohexyl-L-alanine)-(Phe)-(D-Ser)-(D-Tyr)-(Tyr)-(Leu)-(Trp)-(Ser), (Asp)-([beta]-cyclohexyl-L-alanine)-(Phe)-(D-Ser)-(D-Arg)-(Tyr)-(Leu)-(Trp)-(Ser), (Asp)-([beta]-cyclohexyl-L-alanine)-(Phe)-(Ser)-(D-Arg)-(Tyr)-Leu)-(Trp)-(Ser), (D-Asp)-([beta]-cyclohexyl-L-alanine)-(Phe)-(Ser)-(D-Arg)-(Tyr)-Leu)-(Trp)-(Ser), (D-Thr)-([beta]-cyclohexyl-L-alanine)-(Phe)-(D-Ser)-(D-Arg)-(Tyr)-(Leu)-(Trp)-(Ser), (D-Asp)-([beta]-cyclohexyl-L-alanine)-(Phe)-(D-Ser)-(D-Arg)-(Tyr)-(Leu)-([beta]-2-naphthyl-L-alanine)-(Ser), (Asp)-([beta]-cyclohexyl-L-alanine)-(Phe)-(D-Ser)-(Arg)-(Tyr)-(Leu)-(Trp)-(Ser), (Asp)-([beta]-cyclohexyl-L-alanine)-(Phe)-(D-Ser)-(D-Arg)-(Tyr)-(Leu) ([beta]-1-naphthyl-L-alanine)-(Ser), (D-Glu)-([beta]-cyclohexyl-L-alanine)-(Phe)-(D-Ser)-(Tyr)-(Tyr)-(Leu)-(Trp)-(Ser), (Asp)-([beta]-cyclohexyl-L-alanine)-(Phe)-(D-Ser)-(D-Arg)-(Leu)-(Leu)-(Trp)-(D-His), (Asp)-([beta]-cyclohexyl-L-alanine)-(Phe)-(D-Ser)-(D-Arg)-([beta]-cyclohexyl-L-alanine)-(Leu)-(Trp)-(Ile), (Asp)-([beta]-cyclohexyl-L-alanine)-(Phe)-(D-Ser)-(D-Arg)-(Tyr)-(Leu) ([beta]-1-naphthyl-L-alanine)-(D-His), (Asp)-([beta]-cyclohexyl-L-alanine)-(Phe)-(D-Ser)-(D-Arg)-(N-(2,3-dimethoxybenzyl)glycine)-(D-Phe)-(N-(3-indolylethyl)glycine)-(N-(2-methoxyethyl)glycine), (Asp)-([beta]-cyclohexyl-L-alanine)-(Phe)-(D-Ser)-(D-Arg)-(N-(2,3-dimethoxybenzyl)glycine)-(D-Phe)-(N-benzylglycine)-(N-(2 [beta]thoxyethyl)glycine), (Asp)-([beta]-cyclohexyl-L-alanine)-(Phe)-(D-Ser)-(D-Arg)-(N-(2,3-dimethoxybenzyl)glycine)-(D-Phe)-(N-(methylnaphthalyl)glycine)-(N-(2-methoxyethyl)glycine), and (Asp)-([beta]-cyclohexyl-L-alanine)-(Phe)-(D-Ser)-(D-Arg)-(N-(2,3-dimethoxybenzyl)glycine)-(D-Phe)-(N-(2,3-dimethoxybenzyl)glycine)-(Ile), and the radionuclide is selected from the group consisting of 67Cu, 225Ac, 212Pb, 161Tb, and 149Tb, preferably 67Cu, 225Ac, or 212Pb, more preferably 67Cu or 225Ac, most preferred 67Cu.

In a preferred embodiment of the first aspect of the present invention the chelating agent is CB-TE2A and the uPAR binding peptide is (D-Asp)-([beta]-cyclohexyl-L-alanine)-(Phe)-(D-Ser)-(D-Arg)-(Tyr)-(Leu)-(Trp)-(Ser), and the radionuclide is selected from the group consisting of 67Cu, 225Ac, 212Pb, 161Tb, and 149Tb, preferably 67Cu, 225Ac, or 212Pb, more preferably 67Cu or 225Ac, most preferred 67Cu.

### Radionuclides

The group of radionuclides, according to the first aspect of the present invention are characterized by undergoing either beta minus decay or alpha decay the exposure of which damage the cells in e.g. a human body. Thus, those radionuclides (67Cu, 225Ac, 212Pb, 161Tb, and 149Tb) are generally considered therapeutic radionuclides particularly suitable for treatment of cancer.

The peptide/chelator conjugates of the first aspect of the present invention are labeled by reacting the conjugate with selected radionuclide from the group consisting of 67Cu, 225Ac, 212Pb, 161Tb, and 149Tb, e.g. as a metal salt, preferably water soluble. The reaction is carried out by known methods in the art.

Preferred radionuclide for labelling of a uPAR binding peptide conjugate of the first aspect of the present invention is 67Cu, 225Ac, or 212Pb, more preferably 67Cu or 225Ac, most preferred 67Cu.

67Cu most often in its divalent state. 67Cu decays by β (beta minus)-decay. 67Cu has a half-life of 2.6 days rendering it suitable for therapeutic applications especially, in targeted cancer therapy. In context of the present invention, the inventor found 67Cu to show surprisingly good compatibility with the uPAR binding peptide conjugates of the present invention, providing a stably labelled conjugate with high specificity towards its target, thereby illustrating that beta emitting radionuclides, in particular 67Cu, are suitable labelling agents for conjugates of the present invention.

In one embodiment of the first aspect of the present invention the chelating agent in a 67Cu labelled uPAR binding peptide conjugate of the present invention is DOTAM.

In one embodiment of the first aspect of the present invention the chelating agent in a 67Cu labelled uPAR binding peptide conjugate of the present invention is NOTA.

In one embodiment of the first aspect of the present invention the chelating agent in a 67Cu labelled uPAR binding peptide conjugate of the present invention is NODAGA.

In one embodiment of the first aspect of the present invention the chelating agent in a 67Cu labelled uPAR binding peptide conjugate of the present invention is SARCOPHAGINE.

In one embodiment of the first aspect of the present invention the chelating agent in a 67Cu labelled uPAR binding peptide conjugate of the present invention is CB-TE2A.

In a preferred embodiment of the first aspect of the present invention the chelating agent in a 67Cu labelled uPAR binding peptide conjugate of the present invention is DOTA or MeCOSar.

In a more preferred embodiment of the first aspect of the present invention the chelating agent in a 67Cu labelled uPAR binding peptide conjugate of the present invention is DOTA.

In a yet most preferred embodiment of the first aspect of the present invention the radionuclide labelled uPAR binding peptide conjugate has the formula:

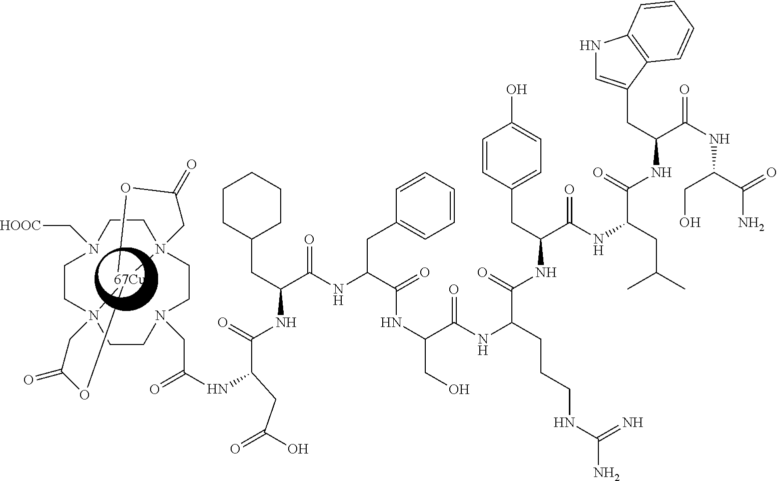

225Ac is an actinide most often in trivalent state. 225Ac decays by alpha decay with a half-life of 9.9 days, rendering it suitable for therapeutic applications especially, in targeted cancer therapy. In context of the present invention, the inventor found 225Ac to show good compatibility with the uPAR binding peptide conjugates of the present invention, providing a stably labelled conjugate with high specificity towards its target, thereby illustrating that alpha emitting radionuclides, in particular 225Ac, are suitable labelling agents for conjugates of the present invention.

In one embodiment of the first aspect of the present invention the chelating agent in a 225Ac labelled uPAR binding peptide conjugate of the present invention is DOTAM.

In one embodiment of the first aspect of the present invention the chelating agent in a 225Ac labelled uPAR binding peptide conjugate of the present invention is NOTA.

In one embodiment of the first aspect of the present invention the chelating agent in a 225Ac labelled uPAR binding peptide conjugate of the present invention is NODAGA.

In one embodiment of the first aspect of the present invention the chelating agent in a 225Ac labelled uPAR binding peptide conjugate of the present invention is SARCOPHAGINE.

In one embodiment of the first aspect of the present invention the chelating agent in a 225Ac labelled uPAR binding peptide conjugate of the present invention is CB-TE2A.

In a preferred embodiment of the first aspect of the present invention the chelating agent in a 225Ac labelled uPAR binding peptide conjugate of the present invention is DOTA or MeCOSar.

In a more preferred embodiment of the first aspect of the present invention the chelating agent in a 225Ac labelled uPAR binding peptide conjugate of the present invention is DOTA.

In a yet more preferred embodiment of the first aspect of the present invention the radionuclide labelled uPAR binding peptide conjugate has the formula:

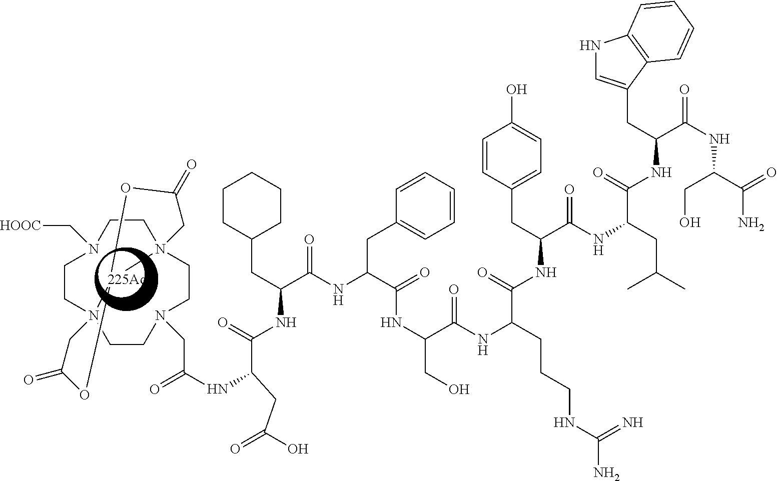

212Pb (212 lead) decays by beta-minus decay with a half-life of 11 hours, generating the alpha-emitting 212Bi (212 Bismuth), thereby providing a dual (i.e. beta-minus and alpha emission induced) therapeutic effect, rendering it suitable for therapeutic applications especially in targeted cancer therapy. In context of the present invention, the inventor found Pb to show good compatibility with the uPAR binding peptide conjugates of the present invention, providing a conjugate with high affinity towards its target, a key prerequisite for therapeutic use of 212Pb labeled conjugates.

In one embodiment of the first aspect of the present invention the chelating agent in a 212Pb labelled uPAR binding peptide conjugate of the present invention is DOTAM.

In one embodiment of the first aspect of the present invention the chelating agent in a 212Pb labelled uPAR binding peptide conjugate of the present invention is NOTA.

In one embodiment of the first aspect of the present invention the chelating agent in a 212Pb labelled uPAR binding peptide conjugate of the present invention is NODAGA.

In one embodiment of the first aspect of the present invention the chelating agent in a 212Pb labelled uPAR binding peptide conjugate of the present invention is SARCOPHAGINE.

In one embodiment of the first aspect of the present invention the chelating agent in a 212Pb labelled uPAR binding peptide conjugate of the present invention is CB-TE2A.

In a preferred embodiment of the first aspect of the present invention the chelating agent in a 212Pb labelled uPAR binding peptide conjugate of the present invention is DOTA or MeCOSar.

In a more preferred embodiment of the first aspect of the present invention the chelating agent in a 212Pb labelled uPAR binding peptide conjugate of the present invention is DOTA.

In a yet more preferred embodiment of the first aspect of the present invention the radionuclide labelled uPAR binding peptide conjugate has the formula:

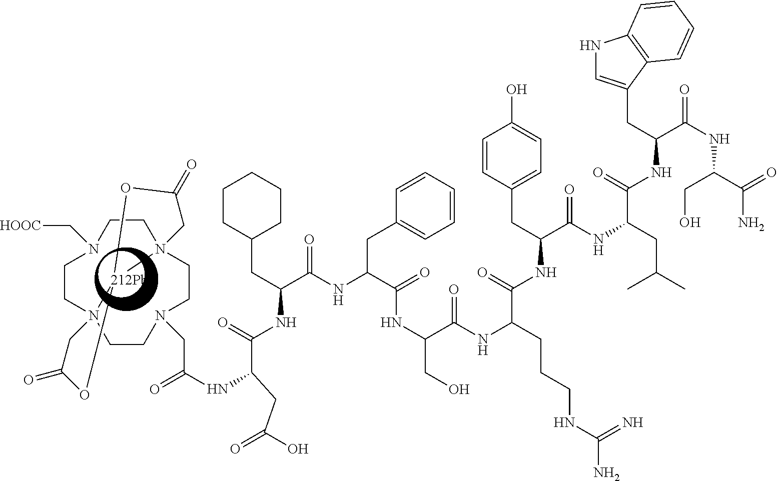

161Tb is a radiolanthanide, most often in trivalent state. 161Tb decays by beta-minus decay with a half-life of 7 days, rendering it suitable for therapeutic applications especially, in targeted cancer therapy.

In one embodiment of the first aspect of the present invention the chelating agent in a 161Tb labelled uPAR binding peptide conjugate of the present invention is DOTAM.

In one embodiment of the first aspect of the present invention the chelating agent in a 161Tb labelled uPAR binding peptide conjugate of the present invention is NOTA.

In one embodiment of the first aspect of the present invention the chelating agent in a 161Tb labelled uPAR binding peptide conjugate of the present invention is NODAGA.

In one embodiment of the first aspect of the present invention the chelating agent in a 161Tb labelled uPAR binding peptide conjugate of the present invention is SARCOPHAGINE.

In one embodiment of the first aspect of the present invention the chelating agent in a 161Tb labelled uPAR binding peptide conjugate of the present invention is CB-TE2A.

In a preferred embodiment of the first aspect of the present invention the chelating agent in a 161Tb labelled uPAR binding peptide conjugate of the present invention is DOTA or MeCOSar.

In a most preferred embodiment of the first aspect of the present invention the chelating agent in a 161Tb labelled uPAR binding peptide conjugate of the present invention is DOTA.

149Tb is a radiolanthanide, most often in trivalent state. 149Tb decays by alpha decay with a half-life of 5.9 days, rendering it suitable for therapeutic applications especially, in targeted cancer therapy.

In one embodiment of the first aspect of the present invention the chelating agent in a 149Tb labelled uPAR binding peptide conjugate of the present invention is DOTAM.

In one embodiment of the first aspect of the present invention the chelating agent in a 149Tb labelled uPAR binding peptide conjugate of the present invention is NOTA.

In one embodiment of the first aspect of the present invention the chelating agent in a 149Tb labelled uPAR binding peptide conjugate of the present invention is NODAGA.

In one embodiment of the first aspect of the present invention the chelating agent in a 149Tb labelled uPAR binding peptide conjugate of the present invention is SARCOPHAGINE.

In one embodiment of the first aspect of the present invention the chelating agent in a 149Tb labelled uPAR binding peptide conjugate of the present invention is CB-TE2A.

In a preferred embodiment of the first aspect of the present invention the chelating agent in a 149Tb labelled uPAR binding peptide conjugate of the present invention is DOTA or MeCOSar.

In a most preferred embodiment of the first aspect of the present invention the chelating agent in a 149Tb labelled uPAR binding peptide conjugate of the present invention is DOTA.

### Diseases

The inventor has shown therapeutic efficacy in three different uPAR expressing cancer diseases. It is generally accepted that therapeutic efficacy across two or three different cancer diseases constitutes a very strong support for therapeutic efficacy in general for cancers expressing the relevant target. Thus, the radionuclide labelled uPAR binding peptide conjugate of the present invention allows for treatment of uPAR expressing cancers, in particular cancer types having high expression of uPAR such as breast cancer, Brain cancer, in particular high grade gliomas, glioblastoma, NSCLC (non-small cell lung cancer), SCLC (small cell lung cancer), Neuroendocrine tumors, Head and neck cancer, HNSCC (Head and Neck squamous cell carcinoma), pancreas cancer, prostate cancer, CRC (Colorectal cancer) and gastric cancer.

One embodiment of the third aspect of the present invention relates to a radionuclide labelled uPAR binding peptide conjugate of the first aspect of the invention, for use in the treatment of breast cancer, Brain cancer, in particular high grade gliomas, glioblastoma, NSCLC (non-small cell lung cancer), SCLC (small cell lung cancer), Neuroendocrine tumors, Head and neck cancer, HNSCC (Head and Neck squamous cell carcinoma), pancreas cancer, prostate cancer, CRC (Colorectal cancer) and gastric cancer, preferably breast cancer, NSCLC, Neuroendocrine tumors, HNSCC, pancreas cancer, prostate cancer, CRC, glioblastoma and gastric cancer, more preferably NSCLC, CRC and glioblastoma, most preferred glioblastoma.

One embodiment of the fourth aspect of the present invention relates to a method of treatment of a uPAR expressing cancer disease by administering to a patient a radionuclide labelled uPAR binding peptide conjugate according to the first aspect, wherein the cancer disease is selected from breast cancer, Brain cancer, in particular high grade gliomas, glioblastoma, NSCLC (non-small cell lung cancer), SCLC (small cell lung cancer), Neuroendocrine tumors, Head and neck cancer, HNSCC (Head and Neck squamous cell carcinoma), pancreas cancer, prostate cancer, CRC (Colorectal cancer) and gastric cancer, preferably NSCLC, Neuroendocrine tumors, HNSCC, pancreas cancer, prostate cancer, CRC, glioblastoma and gastric cancer, more preferably NSCLC, CRC and glioblastoma, most preferred glioblastoma.

A radionuclide labeled uPAR binding peptide conjugate of the present invention where the radionuclide is a beta-emitter, e.g. 67Cu and 161Tb, is prepared to provide a radioactive dose of between about 1-100 MBq in animals, preferable about 30-60 MBq and of 1-20 GBq in humans preferably about 2-10, most preferable around 7.4 GBq, to the individual in accordance with standard radiopharmaceutical dosing determinations. The dose may be administered 1-4 times. A radionuclide labeled uPAR binding peptide conjugate of the present invention where the radionuclide is an alpha-emitter, e.g. 225Ac, 212Pb or 149Tb, is prepared to provide a radioactive dose of between about 1-100 KBq in animals, preferable about 5-60 KBq and of 1-300 MBq in humans preferably about 2-10 MBq, for 225Ac and 50-200 MBq for 212Pb to the individual in accordance with standard radiopharmaceutical dosing determinations. The dose may be administered 1-4 times. The radio labeled peptides may be administered intravenously in any conventional medium for intravenous injection.

### LIST OF EMBODIMENTS

Embodiment 1, A radionuclide labelled uPAR binding peptide conjugate, comprising
a) A uPAR binding peptide
b) A chelating agent suitable for binding radiometals
c) A radionuclide selected from the group consisting of 67Cu, 225Ac, 212Pb, 161Tb, and 149Tb
wherein the peptide is coupled to the radionuclide by the chelating agent.

Preferably said radionuclide labelled uPAR binding peptide conjugate consist of
a) A uPAR binding peptide
b) A chelating agent suitable for binding radiometals
c) A radionuclide selected from the group consisting of 67Cu, 225Ac, 212Pb, 161Tb, and 149Tb
wherein the peptide is coupled to the radionuclide by the chelating agent.

Embodiment 2, The radionuclide labelled peptide conjugate of embodiment 1, wherein the uPAR binding peptide is a monomeric peptide or multimeric peptide wherein the peptide constituent/constituents is/are selected from the group consisting of:

(D-Asp)-([beta]-cyclohexyl-L-alanine)-(Phe)-(D-Ser)-(D-Arg)-(Tyr)-(Leu)-(Trp)-(Ser),
(Ser)-(Leu)-([beta]-cyclohexyl-L-alanine)-(Phe)-(D-Ser)-(Gln)-(Tyr)(Leu)-(Trp)-(Ser),
(D-Glu)-([beta]-cyclohexyl-L-alanine)-(Phe)-(D-Ser)-(D-Tyr)-(Tyr)-(Leu)-(Trp)-(Ser),
(Asp)-([beta]-cyclohexyl-L-alanine)-(Phe)-(D-Ser)-(D-Arg)-(Tyr)-(Leu)-(Trp)-(Ser),
(Asp)-([beta]-cyclohexyl-L-alanine)-(Phe)-(Ser)-(D-Arg)-(Tyr)-Leu)-(Trp)-(Ser),
(D-Asp)-([beta]-cyclohexyl-L-alanine)-(Phe)-(Ser)-(D-Arg)-(Tyr)-Leu)-(Trp)-(Ser),
(D-Thr)-([beta]-cyclohexyl-L-alanine)-(Phe)-(D-Ser)-(D-Arg)-(Tyr)-(Leu)-(Trp)-(Ser),
(D-Asp)-([beta]-cyclohexyl-L-alanine)-(Phe)-(D-Ser)-(D-Arg)-(Tyr)-(Leu)-([beta]-2-
naphthyl-L-alanine)-(Ser),
(Asp)-([beta]-cyclohexyl-L-alanine)-(Phe)-(D-Ser)-(Arg)-(Tyr)-(Leu)-(Trp)-(Ser),
(Asp)-([beta]-cyclohexyl-L-alanine)-(Phe)-(D-Ser)-(D-Arg)-(Tyr)-(Leu)([beta]-1-naphthyl-
L-alanine)-(Ser),
(D-Glu)-([beta]-cyclohexyl-L-alanine)-(Phe)-(D-Ser)-(Tyr)-(Tyr)-(Leu)-(Trp)-(Ser),
(Asp)-([beta]-cyclohexyl-L-alanine)-(Phe)-(D-Ser)-(D-Arg)-(Leu)-(Leu)-(Trp)-(D-His),
(Asp)-([beta]-cyclohexyl-L-alanine)-(Phe)-(D-Ser)-(D-Arg)-([beta]-cyclohexyl-L-alanine)-
(Leu)-(Trp)-(Ile),
(Asp)-([beta]-cyclohexyl-L-alanine)-(Phe)-(D-Ser)-(D-Arg)-(Tyr)-(Leu)([beta]-1-naphthyl-
L-alanine)-(D-His),
(Asp)-([beta]-cyclohexyl-L-alanine)-(Phe)-(D-Ser)-(D-Arg)-(N-(2,3-
dimethoxybenzyl)glycine)-(D-Phe)-(N-(3-indolylethyl)glycine)-(N-(2-
methoxyethyl)glycine),
(Asp)-([beta]-cyclohexyl-L-alanine)-(Phe)-(D-Ser)-(D-Arg)-(N-(2,3-
dimethoxybenzyl)glycine)-(D-Phe)-(N-benzylglycine)-(N-(2[beta]thoxyethyl)glycine),
(Asp)-([beta]-cyclohexyl-L-alanine)-(Phe)-(D-Ser)-(D-Arg)-(N-(2,3-
dimethoxybenzyl)glycine)-(D-Phe)-(N-(methylnaphthalyl)glycine)-(N-(2-
methoxyethyl)glycine), and
(Asp)-([beta]-cyclohexyl-L-alanine)-(Phe)-(D-Ser)-(D-Arg)-(N-(2,3-
dimethoxybenzyl)glycine)-(D-Phe)-(N-(2,3-dimethoxybenzyl)glycine)-(Ile).

Embodiment 3, The radionuclide labelled peptide conjugate of any one of embodiments 1 or 2, wherein the uPAR binding peptide is (D-Asp)-([beta]-cyclohexyl-L-alanine)-(Phe)-(D-Ser)-(D-Arg)-(Tyr)-(Leu)-(Trp)-(Ser)

Embodiment 4, The radionuclide labelled uPAR binding peptide conjugate of any one of embodiments 1 to 3, wherein the chelating agent is selected from the group consisting of DOTA, DOTAM, NOTA, NODAGA, SARCOPHAGINE, CB-TE2A, or derivatives thereof.

Embodiment 5, The radionuclide labelled uPAR binding peptide conjugate of any one of embodiments 1 to 4, wherein the chelating agent is selected from the group consisting of DOTA, DOTAM, SARCOPHAGINE,

Embodiment 6, The radionuclide labelled uPAR binding peptide conjugate of any one of embodiments 1 to 5, wherein the chelating agent is DOTA.

Embodiment 7, The radionuclide labelled uPAR binding peptide conjugate of any one of embodiments 1 to 6 wherein the radionuclide is 67Cu, 225Ac, or 212Pb, preferably 67Cu or 225Ac.

Embodiment 8, The radionuclide labelled uPAR binding peptide conjugate of any one of embodiments 1 to 7 wherein the radionuclide is 67Cu.

Embodiment 9, The radionuclide labelled uPAR binding peptide conjugate of any one of embodiments 1 to 8 having the formula:

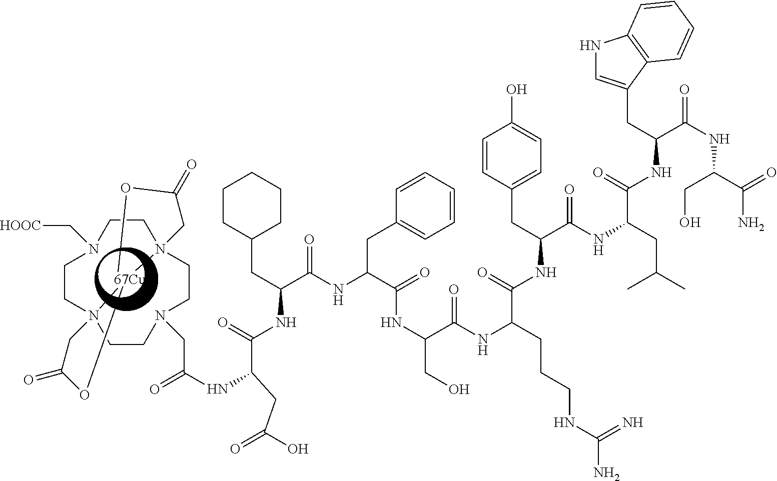

Embodiment 10, The radionuclide labelled uPAR binding peptide conjugate of any one of embodiments 1 to 7 wherein the radionuclide is 225Ac

Embodiment 11, The radionuclide labelled uPAR binding peptide conjugate of any one of embodiments 1 to 7 or 10 having the formula:

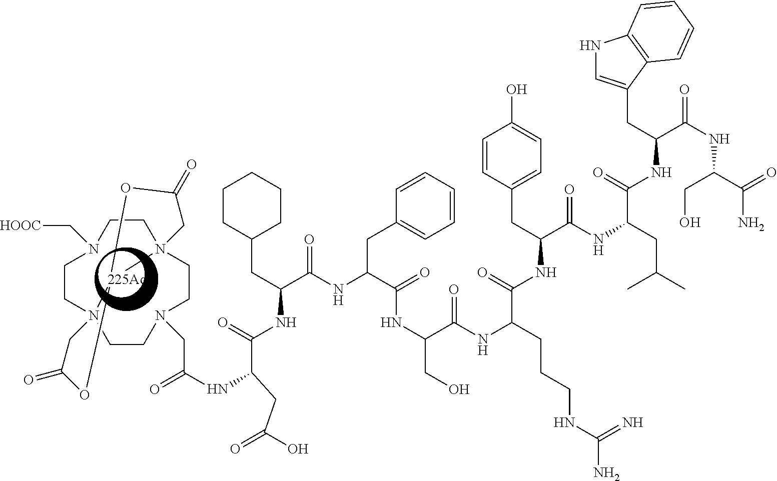

Embodiment 12, The radionuclide labelled uPAR binding peptide conjugate of any one of embodiments 1 to 7 wherein the radionuclide is 212Pb

Embodiment 13, The radionuclide labelled uPAR binding peptide conjugate of any one of embodiments 1 to 7 or 12 having the formula:

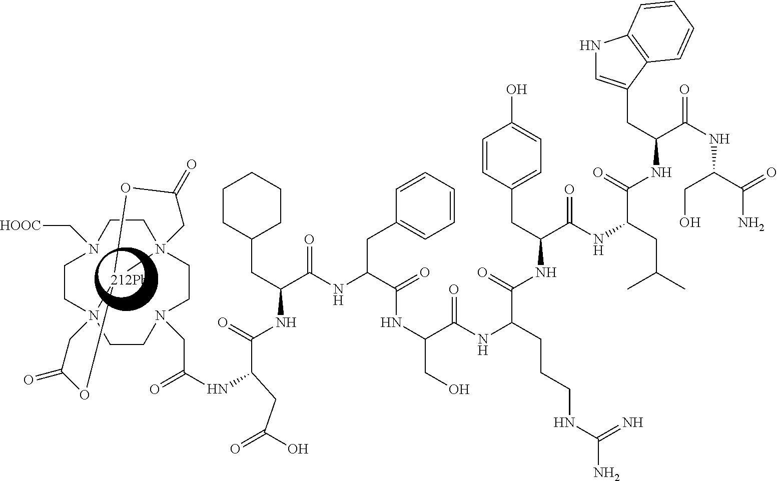

Embodiment 14, The radionuclide labelled uPAR binding peptide conjugate of any one of embodiments 1 to 7, wherein the radionuclide is 67Cu and the chelating agent is DOTA.

Embodiment 15, The radionuclide labelled uPAR binding peptide conjugate of any one of embodiments 1 to 7, wherein the radionuclide is 67Cu and the chelating agent is sarcophagin

Embodiment 16, The radionuclide labelled uPAR binding peptide conjugate of any one of embodiments 1 to 7, wherein the radionuclide is 212Pb and the chelating agent is DOTAM.

Embodiment 17, The radionuclide labelled uPAR binding peptide conjugate of claim of any one of embodiments 1 to 7, wherein the radionuclide is 67Cu and the chelating agent is CB-TE2A

Embodiment 18, A radionuclide labelled uPAR binding peptide conjugate of any one of embodiments 1-17 for use as a medicament.

Embodiment 19, A radionuclide labelled uPAR binding peptide conjugate of any one of embodiments 1-17 for use in the treatment of uPAR expressing cancer.

Embodiment 20, A radionuclide labelled uPAR binding peptide conjugate of any one of embodiments 1-17 for use according to claim 19 wherein said cancer is selected from the group consisting of breast cancer, Brain cancer, in particular high grade gliomas, glioblastoma, NSCLC (non-small cell lung cancer), SCLC (small cell lung cancer), Neuroendocrine tumors, Head and neck cancer, HNSCC (Head and Neck squamous cell carcinoma), pancreas cancer, prostate cancer, CRC (Colorectal cancer), and gastric cancer.

Embodiment 21, A radionuclide labelled uPAR binding peptide conjugate of any one of embodiments 1-17 for use according to embodiment 19 or embodiment 20 wherein said cancer is selected from the group consisting of breast cancer, NSCLC, Neuroendocrine tumors, HNSCC, pancreas cancer, prostate cancer, CRC, glioblastoma, and gastric cancer, preferably NSCLC, CRC, and glioblastoma.

Embodiment 22, A radionuclide labelled uPAR binding peptide conjugate of any one of embodiments 1-17 for use according to any one of embodiments 19-21 wherein said cancer is selected from the group consisting of glioblastoma.

Embodiment 23, Method of treatment of a uPAR expressing cancer disease by administering to a patient a radionuclide labelled uPAR binding peptide conjugate of any one of embodiments 1-17.

Embodiment 24, Method according to embodiment 23, wherein the cancer disease is selected from breast cancer, Brain cancer, in particular high grade gliomas, glioblastoma, NSCLC (non-small cell lung cancer), sclc (small cell lung cancer), Neuroendocrine tumors, Head and neck cancer, HNSCC (Head and Neck squamous cell carcinoma), pancreas cancer, prostate cancer, CRC (Colorectal cancer), and gastric cancer.

Embodiment 25, Method according to embodiments 23 or 24, wherein the cancer disease is selected from NSCLC, Neuroendocrine tumors, HNSCC, pancreas cancer, prostate cancer, CRC, glioblastoma, and gastric cancer, preferably NSCLC, CRC, and glioblastoma.

Embodiment 26, Method according to any one of embodiments 23 to 25, wherein the cancer disease is glioblastoma.

### EXAMPLES

### Example 1: Radiolabeling

### 67Cu Labelling of DOTA-AE105 Conjugate

A radionuclide labelled uPAR binding peptide conjugate according to the present invention was prepared by 67Cu labelling of DOTA-AE105 conjugate.

The compound was labeled with molar activity of 30 and 60 MBq/nmol.

The desired amount of activity from 67Cu stock solution (in 0.01 M HCl) was transferred to an Eppendorf tube, and the activity was measured.

200-300 μL Labeling buffer (0.25 M NH₄OAc, pH 5.2) was mixed with isotope and EtOH was added to the reaction mixture for a 5% volume.

The amount of peptide from the stock is added to reach the desired molar activity, and the reaction volume was adjusted with labeling buffer to a final volume of 500 μL.

The pH in the reaction mixture was measured by indicator paper and was estimated to be 5.0-5.5.

The reaction mixture was stirred at 80° C. for 15 min, at 600 rpm and subsequently cooled to room temperature.

The incorporation of 67Cu into the DOTA-AE105 conjugate was monitored for the crude reaction mixture by radio-TLC and radio-HPLC, and the product (67Cu-DOTA-AE105) was purified by SPE on a tC2 SepPak column by quenching the reaction mixture with DTPA (50 mM, pH 7.0, 2 L) followed by dilution with H₂O to a final volume of 5 mL. The resulting reaction mixture was added to a SPE column, which had been preconditioned with abs. EtOH (5 mL) followed by H₂O (10 mL). After compound addition, the column was washed with H₂O (5 mL) and the product was eluted from the column in EtOH in H₂O (1 mL, 50%, v/v) in fractions of 500 μL. Alternatively, the product can be eluted with 250 μL abs. EtOH. The formulated compound was spiked with unlabeled compound and diluted with formulation buffer (Saline) to reach the molar specific activity (60 or 30 MBq/nmol) and activity concentration of 400 or 200 MBq/mL (60 or 30 MBq/150 μL).

The final product (EOS) was analyzed by radio-TLC and radio-HPLC.

After final injection the labeled compound was analyzed on radio-HPLC followed by stability by radio-HPLC 4 hours after EOS.

The results are shown in table 1, 2 and 3 below.

TABLE 1
67Cu-DOTA-AE105 radiolabeling results

| | 67Cu-DOTA-AE105 | | 67Cu-DOTA-AE105 (30 |
|---|---|---|---|
| | (25 Feb. 2022) | | MBq, 10 Mar. 2022) |
| Incorporation crude, 15 min, radio-HPLC (%) | 91.8 | | 100 |
| Free Cu-67 in crude, 15 min, radio-TLC (%) | 9.1 | | 0 |
| | Group D | Group B + C | Group C |
| Radiochemical purity (RCP) at EOS, radio- | 95.9 | 96.2 | 97.0 |
| HPLC (%) | | | |
| Free Cu-67 at EOS, radio-TLC (%) | 1 | 0 | 0 |
| RCP after final injection (%) | 95.9 | 95.1 | 95.8 |
| Specific activity in final product (MBq/nmol) | 55 | 27.5 | 27.5 |
| Activity concentration in final product | 400 | 200 | 200 |
| (MBq/mL) | | | |
| Peptide concentration in final product | 7.27 | | 7.27 |
| (nmol/mL) | | | |
| EtOH concentration in final product (%) | 4.7 | 3.0 | 24.3 |
| Injection volume (μL) | 150 | | 150 |
| pH (measured by indicator paper) | 5-7 | | 5-7 |
| Appearance | Clear, colorless solution | | Clear, colorless solution |
| Radiochemical identity | 0.24 | 0.24 | 0.21 |
| Stability, 6 H (RCP, %) | 91.2 | — | — |
| Stability, 24 H (RCP, %) | 38.8 | — | — |

TABLE 2
HPLC information for 67Cu-DOTA-AE105

| HPLC | | |
|---|---|---|
| HPLC system | Thermo Scientific Vanquish | |
| Column | Waters XBridge BEH C18, 130 Å, | |
| | 3.5 μm, 4.6 × 50 mm | |
| Mobile phase | A: 0.1% TFA in H₂O, v/v; | |
| | B: 0.1% TFA in MeCN, v/v. | |
| Method | 0-7 min; 5-95% B; 7-8.5 min; | |
| | 95-5% B; 8.5-11 min; 5% B | |
| Flow rate | 1.5 | mL/min |
| UV-detection | 220 | nm |

| | |
|---|---|
| Radio-detector | GABI Nova |
| Column | r.t. |
| temperature | |
| Sample | r.t. |
| temperature | |
| Sample preparation | 2 μL sample in 48 μL 0.1 mM DTPA in H₂O |
| | (crude), undiluted (EOS and stability) |

TABLE 3
TLC information for 67Cu-DOTA-AE105

| TLC | | |
|---|---|---|
| Stationary phase | iTLC-SG | |
| Mobile phase | 0.1M sodium citrate, pH 5.4 | |
| Plate length | 11 | cm |
| Sample volume | 2 μL (crude), 5 μL (EOS) | |
| Detector | miniGita OFA Probe | |
| Acquisition time | 5 | min |

### 225Ac Labelling of DOTA-AE105 Conjugate

A radionuclide labelled uPAR binding peptide conjugate according to the present invention was prepared by 225Ac labelling of DOTA-AE105 conjugate.

The compound was labeled with molar activity of 30 kBq/nmol for the ex vivo biodistribution and 40 kBq/nmol for the efficacy.

The desired amount of activity from 225Ac stock solution (in 0.04 M HCl) was transferred to an Eppendorf tube.

Labeling buffer (1 M ascorbic acid, pH 6.0) was mixed with isotope in the ratio 1:1. It was ensured a compound concentration >90 nmol/mL in reaction mixture.

The amount of peptide from the stock was added to reach the desired molar activity.

The pH in the reaction mixture was measured by indicator paper and was estimated to be 5.7-6.0.

The reaction mixture was stirred at 90° C. for 15 min, at 600 rpm and subsequently cooled to room temperature.

The incorporation of 225Ac into the DOTA-AE105 conjugate was monitored for the crude reaction mixture by radio-TLC. Upon completion, the TLC plate was cut in half and immediately measured twice by gamma counting. The Ac-225 activity of each half was estimated based on the 221Fr and 213Bi peak in the spectrum.

The compound was for the ex vivo biodistribution formulated based on compound amount. For efficacy the compound was formulated based on activity. The formulated compound was diluted with formulation buffer (saline) to reach the compound concentration 6.67 nmol/mL for the ex vivo biodistribution (1 nmol/150 μL) and an activity concentration of 533.33 MBq/mL for the efficacy (80 kBq/150 μL).

The final product (EOS) was analyzed by radio-TLC and radio-HPLC. The TLC plate was cut in half and measured by gamma counting after secular equilibrium. Radio-HPLC fractions of 30 sec interval was collected by automatic fraction collection in an interval of 1 min before/after peak retention time of lanthanum labeled compound reference. Fractions were measured after secular equilibrium by gamma counting.

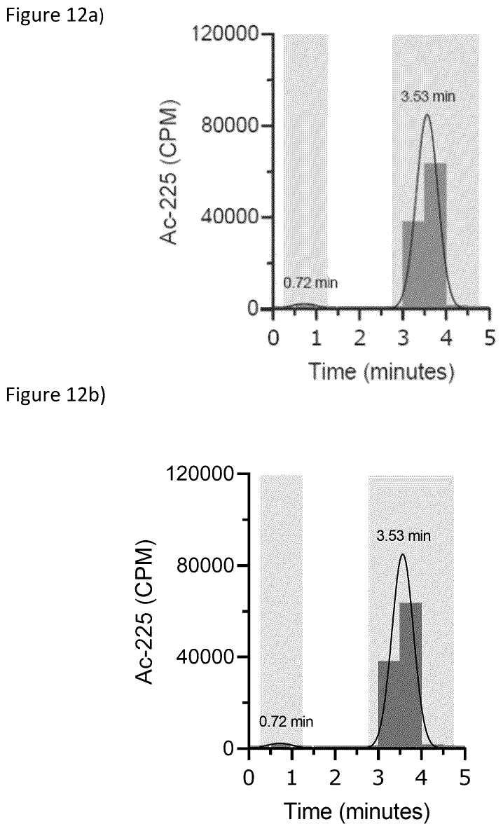

The results are shown in table 1b and 1c below and FIG. 12. FIG. 12a shows Representation of HPLC chromatogram of the 30 kBq/nmol Ac-225-DOTA-AE105 based on gamma counted HPLC fractions. FIG. 12b shows representation of HPLC chromatogram of the 40 kBq/nmol Ac-225-DOTA-AE105 based on gamma counted HPLC fractions.

TABLE 1b
225Ac-DOTA-AE105 radiolabeling results

| | Ac-225-DOTA-AE105 | |
|---|---|---|
| Compounds | (Group A + B) | Comments |
| Specific activity labeled on (kBq/nmol) | 30.0 | |
| Incorporation, crude, radio-TLC (%) | 99.1 | Measured after |
| | | secular equilibrium |
| Purification (Y/N) (if Y, state column) | N | |
| RCP, EOS, radio-HPLC (%) | 96.8 | Fractions |
| | | measured after |
| | | secular equilibrium |
| RCP, EOS, radio-TLC (%) | 98.8 | Measured after |
| | | secular equilibrium |
| Compound concentration, final product | 6.7 | |
| (nmol/mL) | | |
| Activity concentration, final product | 200 | |
| (kBq/mL) | | |
| Molar activity, final product (MBq/nmol) | 29.7 | |
| Injection volume (μL) | 150 | |
| Amount of compound injected (nmol) | 1.0 | |
| Amount of compound injected (μg) | 1.6 | |
| Calculated Activity injected (kBq) | ~30 | |

TABLE 1c
225Ac-DOTA-AE105 radiolabeling results

| | Ac-225-DOTA-AE105 | |
|---|---|---|
| Compounds | (Group C + D + F) | Comments |
| Specific activity labeled on (kBq/nmol) | 40.0 | |
| Incorporation, crude, radio-TLC (%) | 98.3 | Measured after |
| | | secular equilibrium |
| Purification (Y/N) (if Y, state column) | N | |
| RCP, EOS, radio-HPLC (%) | 96.8 | Fractions measured |
| | | after secular |
| | | equilibrium |
| RCP, EOS, radio-TLC (%) | 96.7 | Measured after |
| | | secular equilibrium |
| Compound concentration, final product | 13.6 | |
| (nmol/mL) | | |
| Activity concentration, final product | 533.3 | |
| (kBq/mL) | | |
| Molar activity, final product (MBq/nmol) | 39.4 | |
| Injection volume (μL) | 150 | |
| Amount of compound injected (nmol) | 2.0 | |
| Amount of compound injected (μg) | 3.3 | |
| Calculated Activity injected (kBq) | 80.0 | |

### Example 2: Biodistribution of 67Cu-DOTA-AE105 in Mice

The biodistribution of 67Cu-DOTA-AE105 was evaluated in female NMRI nude mice bearing subcutaneous U87 MG tumors. The U87 MG tumor cell line is a human glioblastoma cell line. Animals were stratified into four groups (A, B, C, D) based on tumor volume and body weight on study day-2. Animals in group A were injected with 26.4±0.4 MBq, animals in group B received 55.1±0.2 MBq, animals in group C received 54.3±0.5 MBq and animals in group D received 55.2±0.2 MBq (mean±SEM). The biodistribution was evaluated using SPECT/CT imaging, 2H, 24H, 48H, 96H and 168H after injection. Furthermore, conventional ex vivo biodistribution was evaluated after final imaging timepoints 96H and 168H.

The experimental setup is summarized in table 4 below.

TABLE 4
Biodistribution in mice study design

| | | | | | Injection | Biodistribution | Biodistribution |
|---|---|---|---|---|---|---|---|
| | | Number | | Specific | volume + | timepoint | timepoint |
| Group | Compound | of mice | Activity | activity | route | (in vivo) | (ex vivo) |
| A | ⁶⁷Cu- | 3 | 30 MBq | 30 | 100 μL, | 2 H, 24 H, 48 H, | NA |
| | DOTA- | | | MBq/nmol | IV | 72 H, 96 H and | |
| | AE105 | | | | | 168 H | |
| B | ⁶⁷Cu- | 3 | 60 MBq | 60 | 100 μL, | 2 H, 24 H, 48 H, | NA |
| | DOTA- | | | MBq/nmol | IV | 72 H, 96 H and | |
| | AE105 | | | | | 168 H | |
| C | ⁶⁷Cu- | 3 | 60 MBq | 60 | 100 μL, | NA | 96 H |
| | DOTA- | | | MBq/nmol | IV | | |
| | AE105 | | | | | | |
| D | ⁶⁷Cu- | 3 | 60 MBq | 60 | 100 μL, | NA | 168 H |
| | DOTA- | | | MBq/nmol | IV | | |
| | AE105 | | | | | | |

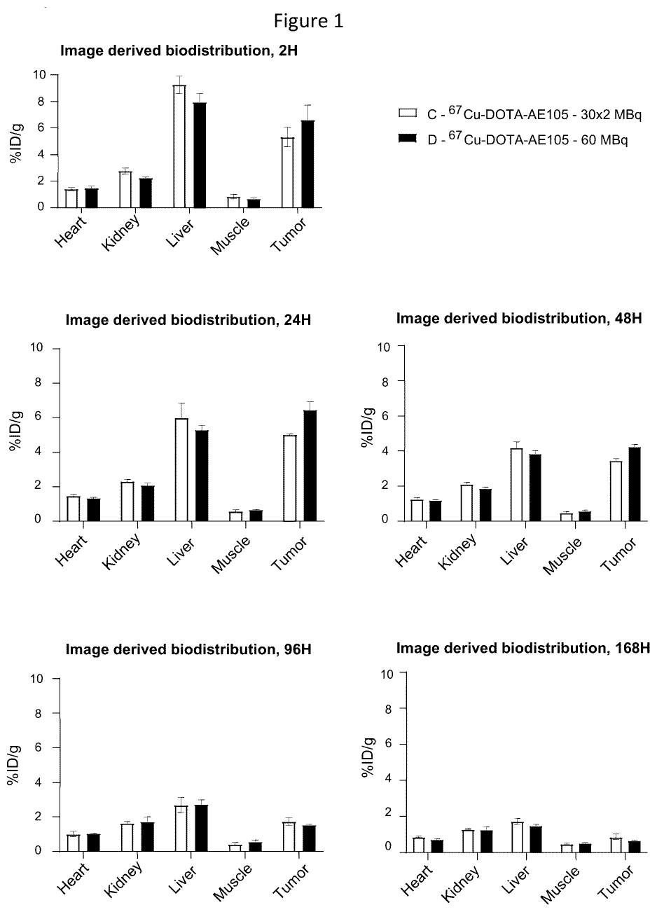

The results are shown in FIG. 1 as percentage injected dose per gram (% ID/g) and are corrected for radioactive decay. The data in FIG. 1 are derived from the in vivo SPECT/CT imaging.

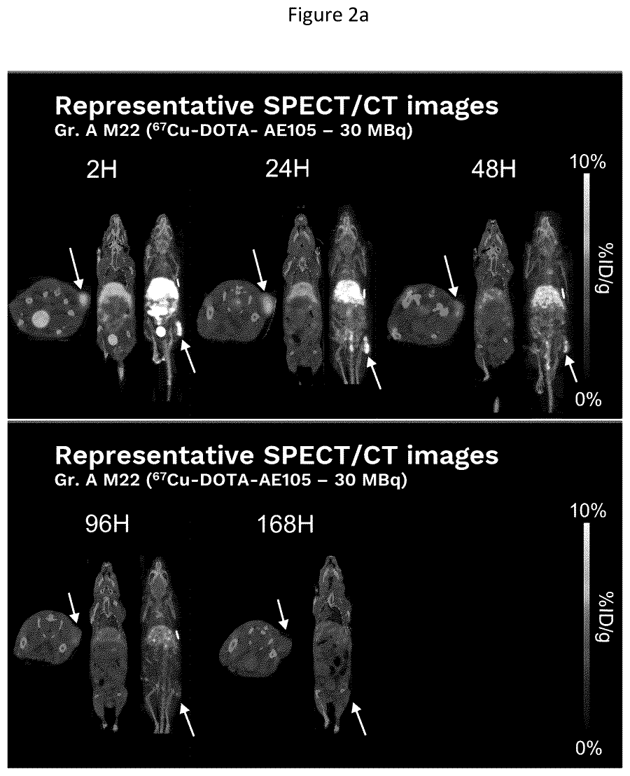

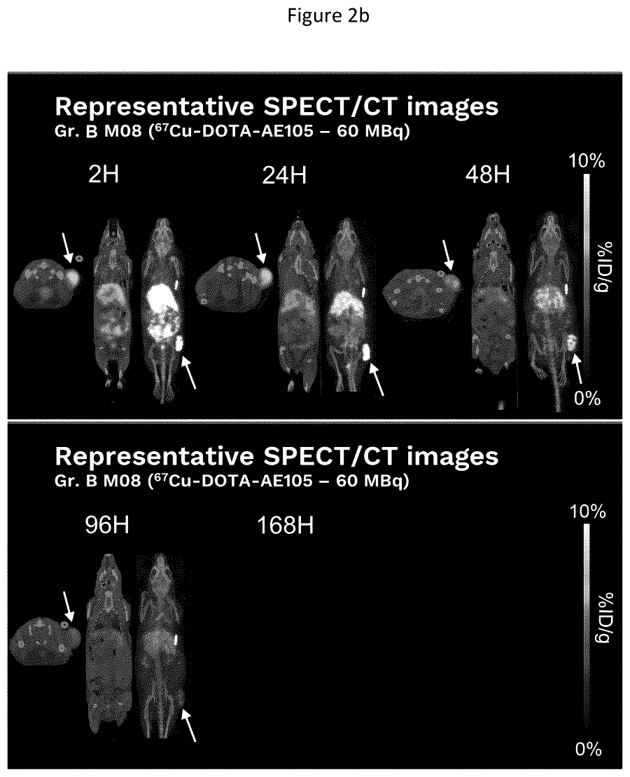

From FIG. 1 it can be appreciated that 67Cu-DOTA-AE105 shows a very high tumor uptake and good tumor retention for 48 hours. Furthermore, 67Cu-DOTA-AE105 shows low uptake in other organs such as heart, kidney, and muscle providing a good safety profile of the drug. FIG. 2 shows axial, coronal, and maximum intensity projection (MIP) images of one representative animal from group A (30 MBq) (FIG. 2a) and group B (60 MBq) (FIG. 2b), and the arrow indicate to location of the tumor. More specifically, FIG. 2a shows representative axial, coronal and MIP images 2H, 24H, 48H, 96H and 168H after injection of approximately 30 MBq 67Cu-DOTA-AE105. The left image is an axial slice intersecting the centre of the tumor while the centre image is a coronal slice. The right image is a MIP image. FIG. 2b shows representative axial, coronal and MIP images 2H, 24H, 48H, 96H and 168H after injection of approximately 60 MBq 67Cu-DOTA-AE105. The left image is an axial slice intersecting the centre of the tumor while the centre image is a coronal slice. The right image is a MIP image.

The results for tumor binding and retention are also summarized in table 5 below from which it can be appreciated that 67Cu-DOTA-AE105 shows a very high tumor uptake and good tumor retention for 48 hours for both 30 MBq and 60 MBq doses, in particular for the 30 MBq dose as can be observed in table 5a.

TABLE 5a
Image-derived data on biodistribution of 30 MBq 67Cu-DOTA-AE105 in
mice with xenograft tumors (U87MG) (group A) reported as percentage
injected dose per gram (% ID/g) corrected for radioactive decay.

| | 67Cu-DOTA-AE105 (30 MBq) | | | | |
|---|---|---|---|---|---|
| | 2 h | 24 h | 48 h | 96 h | 168 h |
| Tumor (% ID/g) | 5.31 ± 0.73 | 5.02 ± 0.08 | 3.44 ± 0.11 | 1.74 ± 0.22 | 0.88 ± 0.15 |
| Tumor retention | 100% | 95% | 80% | 33% | 17% |
| (relative to 2 h) | | | | | |

TABLE 5b
Image-derived data on biodistribution of 60 MBq 67Cu-DOTA-AE105 in
mice with xenograft tumors (U87MG) (group B) reported as percentage
injected dose per gram (% ID/g) corrected for radioactive decay.

| | 67Cu-DOTA-AE105 (60 MBq) | | | | |
|---|---|---|---|---|---|
| | 2 h | 24 h | 48 h | 96 h | 168 h |
| Tumor (% ID/g) | 6.61 ± 1.11 | 6.47 ± 0.50 | 4.24 ± 0.14 | 1.70 ± 0.07 | 0.64 ± 0.05 |
| Tumor retention | 100% | 98% | 64% | 26% | 10% |
| (relative to 2 h) | | | | | |

### Example 3: Binding Specificity

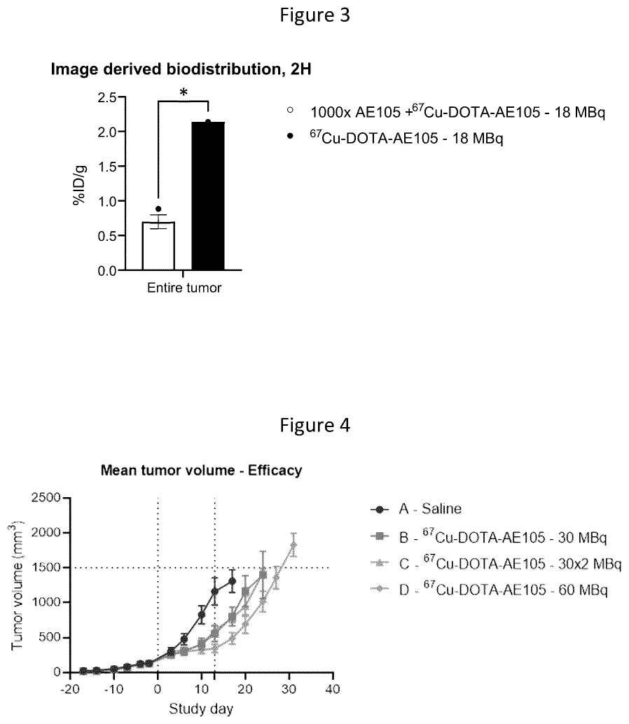

A small study of 4 animals was conducted to evaluate whether it was possible to block the accumulation of 67Cu-DOTA-AE105 by co-administration of unlabeled AE105. Animals were stratified into two groups; a blocking group of three mice and an unblocked group of 1 mouse. The three animals in the blocking group received 1000× excess unlabeled AE105 immediately before injection of 18 MBq 67Cu-DOTA-AE105. The animal in the unblocked group was only injected with 67Cu-DOTA-AE105. The blocking of tumor uptake was evaluated by in vivo (SPECT/CT imaging) biodistribution 2 hours after injection. The results are shown in FIG. 3 where the bars present mean±SEM. From FIG. 3 it can be seen that it was possible to block >50% of the tumor uptake by injecting 1000× excess unlabeled AE105.

### Example 4: Therapeutic Effect in GBM Bearing Xenograft Mice

The treatment efficacy of 67Cu-DOTA-AE105 was tested in female NMRI nude mice bearing subcutaneous U87 MG tumors. The U87 MG tumor cell line is a human glioblastoma cell line.

Animals were stratified into four groups (A, B, C and D) based on tumor volume and body weight on study day-2. The mean inclusion volume was 131.2±8.6 mm³ (range 66.0-258.3 mm³), while the inclusion weight was 29.5±04 g (range 24.2-32.9 g). No significant difference in tumor volume or animal body weight was observed between the groups (one-way ANOVA, p>0.9 and p=0.3, respectively).

Animals were dosed with either vehicle (group A) or 67Cu-DOTA-AE105 (group B, C and D) at different activity levels on study day 0. Animals in group B received 29.6±0.1 MBq, animals in group C received 29.6±0.1 MBq, and animals in group D received 59.5±0.2 MBq. At study day 14 animals in group C received an additional dosing with 30.1±0.1 MBq. Data is presented as dosed measured-residual dose. No decay correction has been applied.

Tumor volume and body weight was monitored twice a week after the first dosing. The efficacy was evaluated over a period of 35 days. The mean tumor volumes for animals included in the study is presented in FIG. 4. Differences in tumor volume were evaluated at the latest timepoint where minimum 50% of animals were alive within each of the compared groups.

At study day 17 all treatment groups had significantly smaller tumor volumes when compared to the vehicle control group (Dunnett's; A vs. B; p<0.03, A vs. C: p=0.02 and A vs. D: p<0.0001).

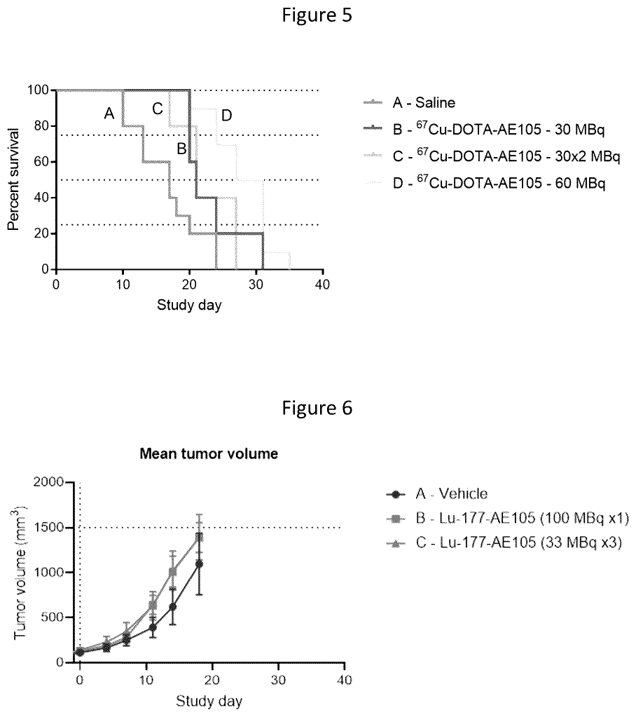

Animals were euthanized according to humane endpoints, which was reached when tumor sizes >1500 mm³. Kaplan-Meier curves of survival data is presented in FIG. 5. The median survival time for animals in the vehicle control group A was 17 days while animals in group B, C and D had a median survival time of 21, 21 and 29 days, respectively.

The body weight did not differ between the groups during the study, indicating that the drug was safe and well tolerated.

### Example 5: Comparison of 67Cu-DOTA-AE105 with 177Lu-DOTA-AE105

The tumor binding and retention as well as treatment efficacy of 67Cu-DOTA-AE105 were compared to that of 177Lu-DOTA-AE105 in a glioblastoma mouse model.

The tumor binding and retention of 177Lu-DOTA-AE105 was evaluated in female NMRI nude mice bearing subcutaneous U87 MG tumors.

Animals were dosed with approximately 50 MBq 177Lu-DOTA-AE105 at study day 0. Tumor binding and retention of 177Lu-DOTA-AE105 was evaluated by SPECT/CT imaging at 0.5H, 2H and 24H after injection. The results for tumor binding and retention of 177Lu-DOTA-AE105 and the data for 67Cu-DOTA-AE105 from example 2 (table 5b) are summarized in table 6 below.

TABLE 6a
Image-derived data on tumor binding and retention
of 50 MBq 177Lu-DOTA-AE105 in mice with xenograft
tumors (U87MG) reported as percentage injected dose
per gram (% ID/g) corrected for radioactive decay.

| | 177Lu-DOTA-AE105 (50 MBq) | | |
|---|---|---|---|
| | 0.5 h | 2 h | 24 h |
| Tumor (% ID/g) | 0.749 ± 0.166 | 0.128 ± 0.011 | 0.098 ± 0.001 |

TABLE 6b
Image-derived data on tumor binding and retention of 60 MBq 67Cu-DOTA-
AE105 in mice with xenograft tumors (U87MG) (group B) reported as percentage
injected dose per gram (% ID/g) corrected for radioactive decay.

| | 67Cu-DOTA-AE105 (60 MBq) | | | | |
|---|---|---|---|---|---|
| | 2 h | 24 h | 48 h | 96 h | 168 h |
| Tumor (% ID/g) | 6.61 ± 1.11 | 6.47 ± 0.50 | 4.24 ± 0.14 | 1.70 ± 0.07 | 0.64 ± 0.05 |

From table 6a it can be appreciated that 177Lu-DOTA-AE105 initially binds the subcutaneous tumor at a level of 0.749 percent injected dose per gram body weight (% ID/g) but rapidly disappears showing limited tumor binding and poor retention at the tumor.

From table 6b it can be appreciated that 67Cu-DOTA-AE105 shows a very high tumor uptake of 6.61% ID/g and good tumor retention for 48 hours.

Furthermore, the efficacy of 177Lu-DOTA-AE105 was tested in female NMRI nude mice bearing subcutaneous U87 MG tumors.

Animals were stratified into three groups (A, B, and C) based on tumor volume and body weight on study day 0. The mean inclusion volume was 135.8±814.6 mm³ (range 15.0-339.0 mm³), while the inclusion weight was 23.6±0.4 g (range 29.4-37.4 g). No significant difference in tumor volume or animal body weight was observed between the groups (one-way ANOVA, p=0.4).

Animals were dosed with either vehicle (group A) or 177Lu-DOTA-AE105 (group B, and C) at different activity levels on study day 0. Animals in group B received 92.1±1.7 MBq, animals in group C received 32.9±0.4 MBq. At study day 4 animals in group C received an additional dosing with 26.7±0.2 MBq and at study day 8 animals in group C were dosed with 30.7±0.2 MBq. Doses are calculated by retracting residual dose from the activity measured before injection. No decay correction has been applied.

Tumor volume and body weight was monitored twice a week after the first dosing. The efficacy was evaluated over a period of 5 weeks after first dosing. Each of the treatment groups were compared to the vehicle control group A.

The mean tumor volumes for animals included in the study is presented in FIG. 6. Differences in tumor volume were evaluated at the latest timepoint where minimum 50% of animals were alive within each of the compared groups.

A comparison in tumor volume between the vehicle control group and the treatment groups at study day 18 showed no significant difference between the groups (one-way ANOVA, Dunnett's, A vs. B; p=0.32 and A vs. C; p=0.84).

### Example 6: Therapeutic Effect of 67Cu-DOTA-AE105 in Glioblastoma and Non-Small Cell Lung Cancer

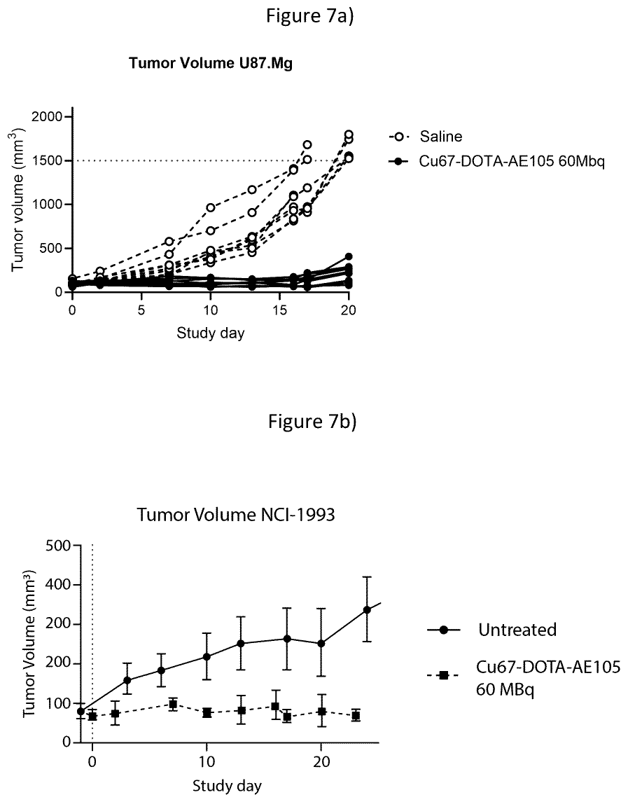

The treatment efficacy of 67Cu-DOTA-AE105 was tested in female NMRI nude mice bearing either subcutaneous U87 MG tumors (see FIG. 7a) or NCI-H1993 tumors (see FIG. 7b). The U87 MG tumor cell line is a human glioblastoma cell line and the NCI-H1993 tumor cell line is a human non-small cell lung cancer (NSCLC) cell line.

In each experiment, animals were stratified into two groups based on tumor volume and body weight on study day-2. No significant difference in tumor volume or animal body weight was observed between the groups.

Animals were either left untreated or dosed with 60 Mbq of 67Cu-DOTA-AE105 at study day 0.

Tumor volume and body weight was monitored twice a week after the drug administration. The efficacy was evaluated over a period of 20 days (glioblastoma study) or 25 days (non-small cell lung cancer study) respectively. The mean tumor volumes for animals included in the non-small cell lung cancer study is presented in FIG. 7b. Differences in tumor volume were evaluated at the latest timepoint where minimum 50% of animals were alive within each of the compared groups.

All treatment groups had significantly smaller tumor volumes when compared to the vehicle control group.

Animals were euthanized according to humane endpoints, which was reached when tumor sizes >1500 mm³.

The body weight did not differ between the groups during the study, indicating that the drug was safe and well tolerated.

It can be observed in FIG. 7 how the tumor growth in both the glioblastoma bearing xenograft mice (FIG. 7a) as well as the NSCLC bearing xenograft mice (FIG. 7b, showing mean tumor volume) was clearly inhibited throughout the study period by administration of a single dose of 60 Mbq of 67Cu-DOTA-AE105 at day 0.

### Example 7: Therapeutic Effect of 67Cu-DOTA-AE105 in Colorectal Cancer

The treatment efficacy of 67Cu-DOTA-AE105 was tested in female NMRI nude mice bearing subcutaneous HT29 tumors. The HT29 tumor cell line is a human colorectal tumor cell line.

Animals were stratified into three groups based on tumor volume and body weight on study day-2. No significant difference in tumor volume or animal body weight was observed between the groups. The first group was left untreated, the second group was treated with 60 MBq at study day 0 and the third group was treated with 60 MBq at study day 0 and again at study day 7.

The xenograft tumors were harvested at day 0 (baseline), day 2 and day 10 after treatment and subjected to analysis by flow cytometry and immunohistochemistry.

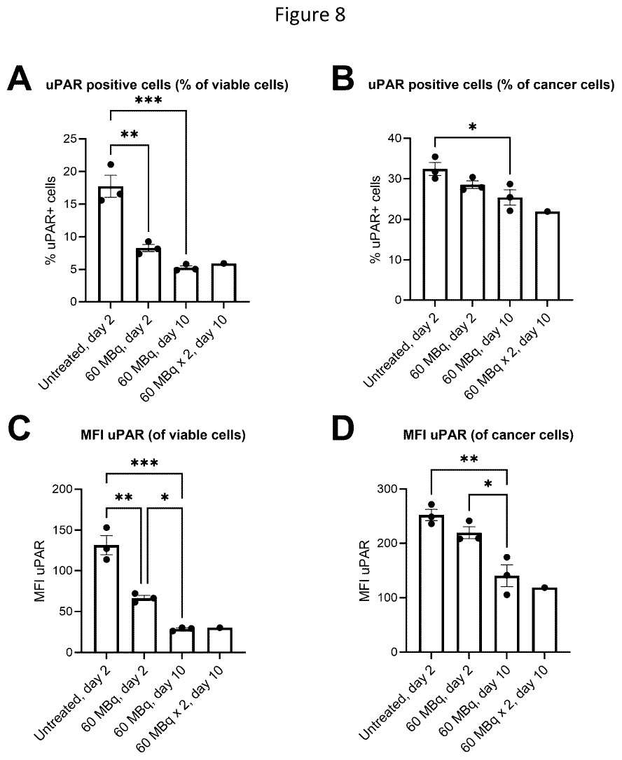

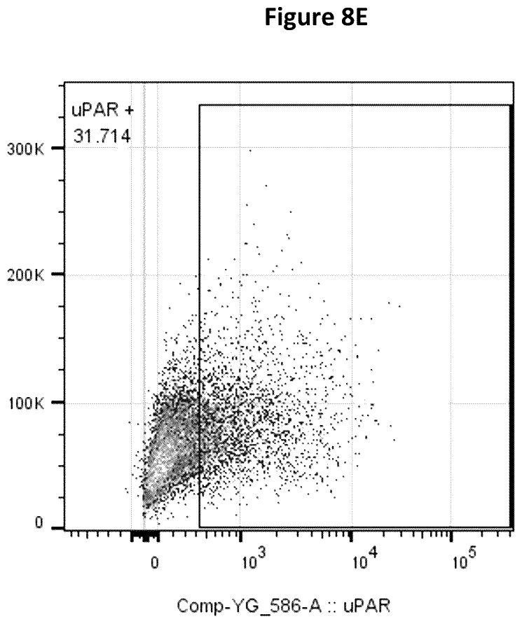

The flow cytometry was performed using standard methods known to the skilled person. The uPAR positive cells were labeled using a fluorescent uPAR antibody and the number of viable uPAR positive cells in the xenograft tumors were determined. The results are presented in FIG. 8. FIG. 8a show the percentage of viable cells that were uPAR positive. FIG. 8b shows the percentage of viable cancer cells that were uPAR positive. FIG. 8c shows the mean fluorescence intensity (MFI) of the uPAR marker (fluorescent antibody) for viable cells. FIG. 8d shows the MFI of the uPAR marker for cancer cells. FIG. 8e shows the cutoff applied for defining uPAR positive cells in the flow cytometry analysis.

The immunohistochemistry was performed using standard preparatory methods of sliced tumor tissue. The tumor tissue samples were stained for DNA double strand breaks (using a yH2AX specific antibody), apoptosis (using cCASP-3, c and p specific antibodies), and uPAR (using a uPAR specific antibody) respectively.

From FIG. 8 it can be appreciated that uPAR positive cancer cells are diminishing over a 10 day period after treatment with 60 MBq. Furthermore, it can be appreciated that continuous killing of uPAR positive cancer cells can be observed over time and further increases after two doses of the study drug.

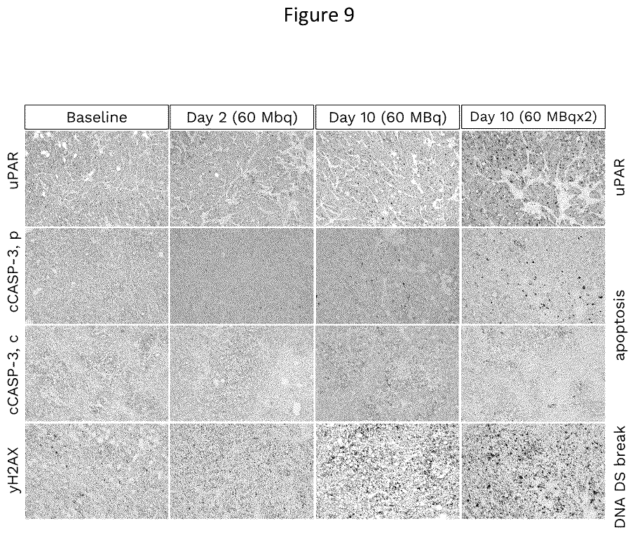

From the immunohistochemistry in FIG. 9 it can be appreciated that treatment with 67Cu-DOTA-AE105 induce apoptosis as well as DNA double strand breaks in human colorectal cancer.

### Example 8: Biodistribution of 225Ac-DOTA-AE105

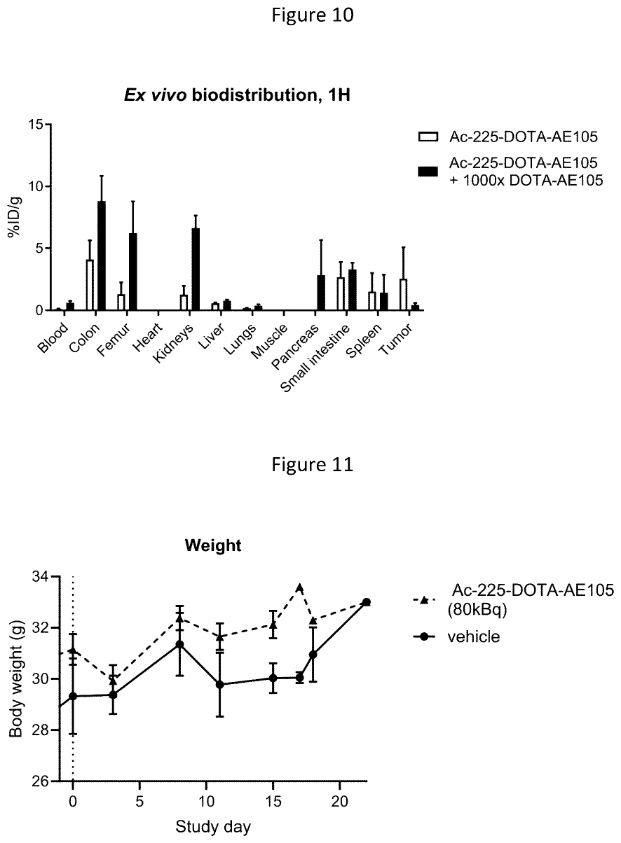

The biodistribution of 225Ac-DOTA-AE105 was evaluated in female NMRI nude mice bearing subcutaneous U87 MG tumors. The U87 MG tumor cell line is a human glioblastoma cell line. Animals were stratified into two groups (A and B) based on tumor volume and body weight on study day-2. Animals in group A were injected with 30 kBq of 225Ac-DOTA-AE105 having a specific activity of 30 kBq/nmol, animals in group B received a thousand-fold dose of DOTA-AE105 (cold construct) immediately before receiving 30 kBq of 225Ac-DOTA-AE105 having a specific activity of 30 kBq/nmol. The biodistribution was evaluated using conventional ex vivo biodistribution 1 hour (1H) after injection. The results are shown in FIG. 10 as percentage injected dose per gram (% ID/g) and are corrected for radioactive decay.

From FIG. 10 it can be appreciated that the biodistribution with the alpha-emitting uPAR-targeting therapeutic compound 225Ac-DOTA-AE105 shows a high uptake (3% ID/g) in human xenograft glioblastoma tumors (U87MG). Blocking with cold DOTA-AE105 demonstrates that the tumor-binding of 225Ac-DOTA-AE105 is specific as illustrated by the much lower uptake in tumors in group B.

### Example 9: Tolerability and Safety of 225AC-DOTA-AE105

The tolerability and safety of 225-AC-AE105 was evaluated in female NMRI nude mice. The animals were stratified into two groups based on body weight on study day-2.

Animals in control group received vehicle (negative control) at study day 0 and animals in treatment group received 80 kBq of 225Ac-DOTA-AE105 at study day 0. The body weight of the animals was monitored over a period of 25 days and the results are shown in FIG. 11.

From FIG. 11 it can be appreciated that the body weight of the animals remained stable during the study, indicating that the alpha-emitting uPAR targeting therapy administered in a dose of 80 kBq was well-tolerated.

### Example 10: Therapeutic Effect of 225Ac-DOTA-AE105 in Glioblastoma

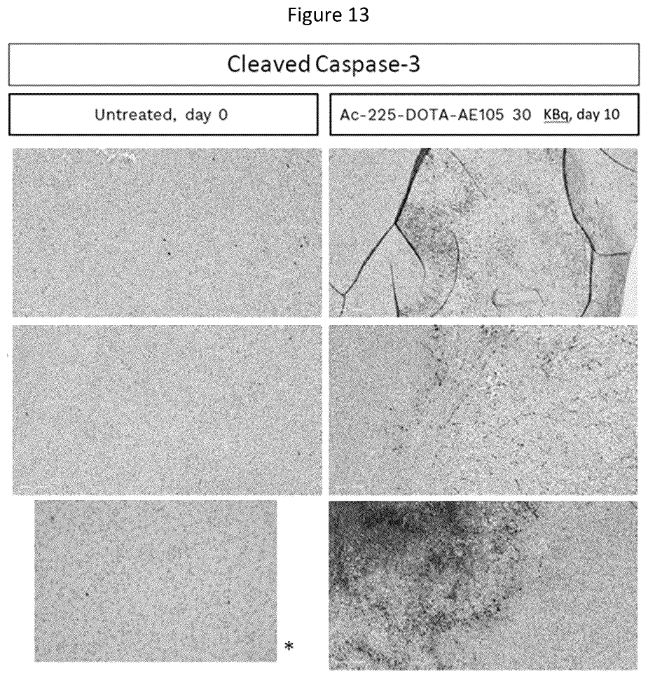

Efficacy of 225Ac-DOTA-AE105 was evaluated in mice with implanted glioblastoma xenograft tumors (U87.MG). A single dose of 30 KBq 225Ac-DOTA-AE105 was administered to the mice and Ex vivo analysis of apoptosis was performed at day 10 by immunohistochemistry staining for caspase-3, a marker of apoptosis. The results are shown in FIG. 13 and clearly demonstrates the induction of apoptosis in the active group (who received 225Ac-DOTA-AE105 treatment) whereas no such induction is seen in the vehicle (control) group.

### Example 11: UPAR Binding Characterization of 212Pb-DOTA-AE105

Characterization of the binding properties of lead (Pb) labelled DOTA-AE105 to its target i.e. uPAR was measured by Surface Plasmon Resonance (SPR) and expressed as the binding constant (K_{D}), which is a measure of affinity of the conjugate towards its target.

Characterizing the interaction between a target protein (in this case uPAR) and a ligand by SPR is a well-known method for the skilled person.

The target protein uPAR was captured, via a protein tag, onto a sensor chip and the test compound (Pb2+-DOTA-AE105 and Cu2+-DOTA-AE105 respectively) were dissolved in running buffer (10 mM HEPES buffer at pH 7.4, 150 mM NaCl, 50 mM EDTA+0.05% Tween-20) and prepared in 5-point concentration series with three-fold dilution with top concentration around 500 nM of the test compound. The assay was conducted at 18 degrees Celsius.

The results are shown in table 7 below. From the table below it can be appreciated that for the cold version of 212Pb-DOTA-AE105 the measured binding constant (K_{D}) was 17.4 nM compared to 182.0 nM for the corresponding cold version of 67Cu-DOTA-AE105. Accordingly, affinity was more than 10 fold higher for Pb2+-DOTA-AE105 demonstrating that even better therapy efficacy is to be expected with 212Pb-DOTA-AE105. The difference in K_{D} was mainly due to a much slower off rate of Pb2+-DOTA-AE105, which is what is needed for radionuclides to stay on the target and be effective e.g. in therapeutic use.

TABLE 7
SPR results

| Ligand | Chelator | Metal | SPR K(on) | SPR K(off) | SPR K(D) |
|---|---|---|---|---|---|
| AE105 | DOTA | Cu2+ | 0.110 | 19.95 | 182.0 |
| AE105 | DOTA | Pb2+ | 0.102 | 1.78 | 17.4 |

1-15. (canceled)

16. A radionuclide labelled uPAR binding peptide conjugate, comprising
a uPAR binding peptide
a chelating agent suitable for binding radiometals
a radionuclide selected from the group consisting of 67Cu, 225Ac, 212Pb, 161Tb, and 149Tb
wherein the peptide is coupled to the radionuclide by the chelating agent, and wherein the uPAR binding peptide is a monomeric peptide or multimeric peptide wherein the peptide constituent/constituents is/are selected from the group consisting of:
(D-Asp)-([beta]-cyclohexyl-L-alanine)-(Phe)-(D-Ser)-(D-Arg)-(Tyr)-(Leu)-(Trp)-
(Ser),
(Ser)-(Leu)-([beta]-cyclohexyl-L-alanine)-(Phe)-(D-Ser)-(Gln)-(Tyr)(Leu)-(Trp)-
(Ser),
(D-Glu)-([beta]-cyclohexyl-L-alanine)-(Phe)-(D-Ser)-(D-Tyr)-(Tyr)-(Leu)-(Trp)-
(Ser),
(Asp)-([beta]-cyclohexyl-L-alanine)-(Phe)-(D-Ser)-(D-Arg)-(Tyr)-(Leu)-(Trp)-(Ser),
(Asp)-([beta]-cyclohexyl-L-alanine)-(Phe)-(Ser)-(D-Arg)-(Tyr)-Leu)-(Trp)-(Ser),
(D-Asp)-([beta]-cyclohexyl-L-alanine)-(Phe)-(Ser)-(D-Arg)-(Tyr)-Leu)-(Trp)-(Ser),
(D-Thr)-([beta]-cyclohexyl-L-alanine)-(Phe)-(D-Ser)-(D-Arg)-(Tyr)-(Leu)-(Trp)-
(Ser),
(D-Asp)-([beta]-cyclohexyl-L-alanine)-(Phe)-(D-Ser)-(D-Arg)-(Tyr)-(Leu)-([beta]-2-
naphthyl-L-alanine)-(Ser),
(Asp)-([beta]-cyclohexyl-L-alanine)-(Phe)-(D-Ser)-(Arg)-(Tyr)-(Leu)-(Trp)-(Ser),
(Asp)-([beta]-cyclohexyl-L-alanine)-(Phe)-(D-Ser)-(D-Arg)-(Tyr)-(Leu)([beta]-1-
naphthyl-L-alanine)-(Ser),
(D-Glu)-([beta]-cyclohexyl-L-alanine)-(Phe)-(D-Ser)-(Tyr)-(Tyr)-(Leu)-(Trp)-(Ser),
(Asp)-([beta]-cyclohexyl-L-alanine)-(Phe)-(D-Ser)-(D-Arg)-(Leu)-(Leu)-(Trp)-(D-
His),
(Asp)-([beta]-cyclohexyl-L-alanine)-(Phe)-(D-Ser)-(D-Arg)-([beta]-cyclohexyl-L-
alanine)-(Leu)-(Trp)-(Ile),
(Asp)-([beta]-cyclohexyl-L-alanine)-(Phe)-(D-Ser)-(D-Arg)-(Tyr)-(Leu)([beta]-1-
naphthyl-L-alanine)-(D-His),
(Asp)-([beta]-cyclohexyl-L-alanine)-(Phe)-(D-Ser)-(D-Arg)-(N-(2,3-
dimethoxybenzyl)glycine)-(D-Phe)-(N-(3-indolylethyl)glycine)-(N-(2-
methoxyethyl)glycine),
(Asp)-([beta]-cyclohexyl-L-alanine)-(Phe)-(D-Ser)-(D-Arg)-(N-(2,3-
dimethoxybenzyl)glycine)-(D-Phe)-(N-benzylglycine)-(N-
(2[beta]thoxyethyl)glycine),
(Asp)-([beta]-cyclohexyl-L-alanine)-(Phe)-(D-Ser)-(D-Arg)-(N-(2,3-
dimethoxybenzyl)glycine)-(D-Phe)-(N-(methylnaphthalyl)glycine)-(N-(2-
methoxyethyl)glycine),and
(Asp)-([beta]-cyclohexyl-L-alanine)-(Phe)-(D-Ser)-(D-Arg)-(N-(2,3-
dimethoxybenzyl)glycine)-(D-Phe)-(N-(2,3-dimethoxybenzyl)glycine)-(Ile).

17. The radionuclide labelled peptide conjugate of claim 16, wherein the uPAR binding peptide is (D-Asp)-([beta]-cyclohexyl-L-alanine)-(Phe)-(D-Ser)-(D-Arg)-(Tyr)-(Leu)-(Trp)-(Ser)

18. The radionuclide labelled uPAR binding peptide conjugate of claim 16, wherein the chelating agent is selected from the group consisting of DOTA, DOTAM, NOTA, NODAGA, SARCOPHAGINE, CB-TE2A, or derivatives thereof.

19. The radionuclide labelled uPAR binding peptide conjugate of claim 16, wherein the chelating agent is selected from the group consisting of DOTA, DOTAM, SARCOPHAGINE,

20. The radionuclide labelled uPAR binding peptide conjugate of claim 16, wherein the chelating agent is DOTA.

21. The radionuclide labelled uPAR binding peptide conjugate of claim 16 wherein the radionuclide is 67Cu, 225Ac, or 212Pb.

22. The radionuclide labelled uPAR binding peptide conjugate of claim 16 wherein the radionuclide is 67Cu.

23. The radionuclide labelled uPAR binding peptide conjugate of claim 16 having the formula:

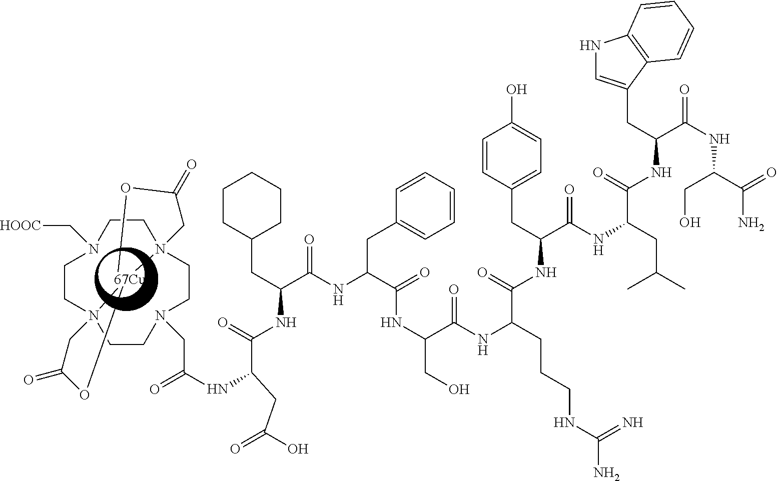

24. The radionuclide labelled uPAR binding peptide conjugate of claim 16, wherein the radionuclide is 67Cu and the chelating agent is DOTA.

25. A radionuclide labelled uPAR binding peptide conjugate of claim 16 for use as a medicament.

26. A radionuclide labelled uPAR binding peptide conjugate of for use according to claim 25 in the treatment of uPAR expressing cancer.

27. A radionuclide labelled uPAR binding peptide conjugate for use according to claim 26 wherein said cancer is selected from the group consisting of breast cancer, Brain cancer, in particular high grade gliomas, glioblastoma, NSCLC (non-small cell lung cancer), SCLC (small cell lung cancer), Neuroendocrine tumors, Head and neck cancer, HNSCC (Head and Neck squamous cell carcinoma), pancreas cancer, prostate cancer, CRC (Colorectal cancer), and gastric cancer.

28. A radionuclide labelled uPAR binding peptide conjugate for use according to claim 26 wherein said cancer is selected from the group consisting of breast cancer, NSCLC, Neuroendocrine tumors, HNSCC, pancreas cancer, prostate cancer, CRC, glioblastoma, and gastric cancer.

29. A radionuclide labelled uPAR binding peptide conjugate for use according to claim 26 wherein said cancer is selected from the group consisting of glioblastoma.


SEQUENCE LISTING (18 sequences)

SEQ ID NO: 1 - protein, 9 aa, synthetic construct
  SITE 2: note = [beta]-cyclohexyl-L-alanine
  SITE 1: note = D-aspartic acid
  SITE 4: note = D-serine
  SITE 5: note = D-arginine
DXFSRYLWS

SEQ ID NO: 2 - protein, 10 aa, synthetic construct
  SITE 3: note = [beta]-cyclohexyl-L-alanine
  SITE 5: note = D-serine
SLXFSQYLWS

SEQ ID NO: 3 - protein, 9 aa, synthetic construct
  SITE 1: note = D-glutamic acid
  SITE 2: note = [beta]-cyclohexyl-L-alanine
  SITE 4: note = D-serine
  SITE 5: note = D-tyrosine
EXFSYYLWS

SEQ ID NO: 4 - protein, 9 aa, synthetic construct
  SITE 2: note = [beta]-cyclohexyl-L-alanine
  SITE 4: note = D-serine
  SITE 5: note = D-arginine
DXFSRYLWS

SEQ ID NO: 5 - protein, 9 aa, synthetic construct
  SITE 2: note = [beta]-cyclohexyl-L-alanine
  SITE 5: note = D-arginine
DXFSRYLWS

SEQ ID NO: 6 - protein, 9 aa, synthetic construct
  SITE 1: note = D-aspartic acid
  SITE 2: note = [beta]-cyclohexyl-L-alanine
  SITE 5: note = D-arginine
DXFSRYLWS

SEQ ID NO: 7 - protein, 9 aa, synthetic construct
  SITE 1: note = D-threonine
  SITE 2: note = [beta]-cyclohexyl-L-alanine
  SITE 4: note = D-serine
  SITE 5: note = D-arginine
TXFSRYLWS

SEQ ID NO: 8 - protein, 9 aa, synthetic construct
  SITE 1: note = D-aspartic acid
  SITE 2: note = [beta]-cyclohexyl-L-alanine
  SITE 4: note = D-serine
  SITE 5: note = D-arginine
  SITE 8: note = [beta]-2-naphthyl-L-alanine
DXFSRYLXS

SEQ ID NO: 9 - protein, 9 aa, synthetic construct
  SITE 2: note = [beta]-cyclohexyl-L-alanine
  SITE 4: note = D-serine
DXFSRYLWS

SEQ ID NO: 10 - protein, 9 aa, synthetic construct
  SITE 2: note = [beta]-cyclohexyl-L-alanine
  SITE 4: note = D-serine
  SITE 5: note = D-arginine
  SITE 8: note = [beta]-1-naphthyl-L-alanine
DXFSRYLXS

SEQ ID NO: 11 - protein, 9 aa, synthetic construct
  SITE 1: note = D-glutamic acid
  SITE 2: note = [beta]-cyclohexyl-L-alanine
  SITE 4: note = D-serine
EXFSYYLWS

SEQ ID NO: 12 - protein, 9 aa, synthetic construct
  SITE 2: note = [beta]-cyclohexyl-L-alanine
  SITE 4: note = D-serine
  SITE 5: note = D-arginine
  SITE 9: note = D-histidine
DXFSRLLWH

SEQ ID NO: 13 - protein, 9 aa, synthetic construct
  SITE 2: note = [beta]-cyclohexyl-L-alanine
  SITE 4: note = D-serine
  SITE 5: note = D-arginine
  SITE 6: note = [beta]-cyclohexyl-L-alanine
DXFSRXLWI

SEQ ID NO: 14 - protein, 9 aa, synthetic construct
  SITE 2: note = [beta]-cyclohexyl-L-alanine
  SITE 4: note = D-serine
  SITE 5: note = D-arginine
  SITE 8: note = [beta]-1-naphthyl-L-alanine
  SITE 9: note = D-histidine
DXFSRYLXH

SEQ ID NO: 15 - protein, 9 aa, synthetic construct
  SITE 2: note = [beta]-cyclohexyl-L-alanine
  SITE 4: note = D-serine
  SITE 5: note = D-arginine
  SITE 6: note = N-(2,3-dimethoxybenzyl)glycine
  SITE 7: note = D-phenylalanine
  SITE 8: note = N-(3-indolylethyl)glycine
  SITE 9: note = N-(2-methoxyethyl)glycine
DXFSRXFXX

SEQ ID NO: 16 - protein, 9 aa, synthetic construct
  SITE 2: note = [beta]-cyclohexyl-L-alanine
  SITE 4: note = D-serine
  SITE 5: note = D-arginine
  SITE 6: note = N-(2,3-dimethoxybenzyl)glycine
  SITE 7: note = D-phenylalanine
  SITE 8: note = N-benzylglycine
  SITE 9: note = N-(2[beta]thoxyethyl)glycine
DXFSRXFXX

SEQ ID NO: 17 - protein, 9 aa, synthetic construct
  SITE 2: note = [beta]-cyclohexyl-L-alanine
  SITE 4: note = D-serine
  SITE 5: note = D-arginine
  SITE 6: note = N-(2,3-dimethoxybenzyl)glycine
  SITE 7: note = D-phenylalanine
  SITE 8: note = N-(methylnaphthalyl)glycine
  SITE 9: note = N-(2-methoxyethyl)glycine
DXFSRXFXX

SEQ ID NO: 18 - protein, 9 aa, synthetic construct
  SITE 2: note = [beta]-cyclohexyl-L-alanine
  SITE 4: note = D-serine
  SITE 5: note = D-arginine
  SITE 6: note = N-(2,3-dimethoxybenzyl)glycine
  SITE 7: note = D-phenylalanine
  SITE 8: note = N-(2,3-dimethoxybenzyl)glycine
DXFSRXFXI